(19) United States
(12) Patent Application Publication
(10) Pub. No.: US 2026/0355495 A1
(43) Pub. Date: Nov. 12, 2026

(54) SAFE DISCONNECT INTRA-MEDICAL TUBING CONNECTOR ASSEMBLY

(71) Applicant: ELCAM MEDICAL A.C.A.L., Merom Hagalil (IL)

(72) Inventors: Tzafrir KEYNAN, Upper Galilee (IL); Ziv NAFTALOVITZ, West Galilee (IL); Gilad MERMELSHTEIN, Misgav-Am (IL)

(21) Appl. No.: 19/129,160

(22) PCT Filed: Nov. 10, 2023
(86) PCT No.: PCT/IL2023/051162

§ 371 Date: May 12, 2025

Related U.S. Application Data
(60) Provisional application No. 63/424,880, filed on Nov. 12, 2022.

(51) Int. Cl. A61M 39/26; A61M 39/10

(52) U.S. Cl. CPC A61M 39/26; A61M 39/1011; A61M 2039/1027; A61M 2039/1061

(57) ABSTRACT

An automatically bidirectionally-sealable safe disconnect intra-medical tubing connector assembly including first and second luer-actuated valve assemblies configured for connection to opposite intermediate ends of a medical tube and configured to be connected to each other in a snap-fit manner and arranged for tensile force responsive disconnection; the first luer-actuated valve assembly includes a cannula defining a fluid flow path and a first seal element retained within a shuttle element, the shuttle element is configured to be axially displaceable relative to the cannula and further configured to be biased forwardly by a biasing element; the second luer-actuated valve assembly includes a second seal element.

### REFERENCE TO RELATED APPLICATIONS

Reference is hereby made to PCT Patent application Serial No. PCT/IL2019/050233, filed Mar. 4, 2019 and entitled “SAFE DISCONNECT INTRA-MEDICAL TUBING CONNECTOR ASSEMBLY”, and to PCT Patent application Serial No. PCT/IL2019/050981, filed Sep. 3, 2019 and entitled “SAFE DISCONNECT INTRA-MEDICAL TUBING CONNECTOR ASSEMBLY”, the disclosures of which are hereby incorporated by reference in their entirety.

Reference is additionally made to U.S. Provisional patent application Ser. No. 63/424,880, filed Nov. 12, 2022 and entitled “SAFE DISCONNECT INTRA-MEDICAL TUBING CONNECTOR ASSEMBLY”, the disclosure of which is hereby incorporated by reference in its entirety and priority of which is hereby claimed pursuant to 37 CFR 1.78(a) (4) and (5)(i).

### FIELD OF THE INVENTION

The present invention relates to medical access devices and more particularly to break-away connectors for medical tubing.

### BACKGROUND OF THE INVENTION

Various medical tubes are used in treatment of patients, such as intravenous catheters, feeding tubes, dialysis tubes, enteral feeding, foley catheters. It is appreciated that these medical tubes are generally flexible, thus allowing the patient a range of movement.

For instance, medicaments may be administered to a patient through an IV line, which is connected to a needle, which is in turn fixed to an injection site. An additional example is a catheter which is inserted into a desired treatment location within the body of the patient.

During movement of the patient undergoing treatment, the medical tubes are subject to certain tensile forces that may cause dislocation of the catheter or of the needle. Additionally, certain tensile forces, greater than a pre-determined threshold, may cause spillage of the medicaments being administered to the patient or the patient's body fluids and thus expose the patient to a risk of infection.

### SUMMARY OF THE INVENTION

The present invention seeks to provide an improved safe disconnect intra-medical tubing connector assembly.

There is thus provided in accordance with an embodiment of the present invention an automatically bidirectionally-sealable safe disconnect intra-medical tubing connector assembly comprising first and second luer-actuated valve assemblies configured for connection to opposite intermediate ends of a medical tube and configured to be connected to each other in a snap-fit manner and arranged for tensile force responsive disconnection; the first luer-actuated valve assembly comprises a cannula defining a fluid flow path and a first seal element retained within a shuttle element, wherein the shuttle element is configured to be axially displaceable relative to the cannula and further configured to be biased forwardly by a biasing element; the second luer-actuated valve assembly comprises a second seal element.

Preferably, connection of the first luer-actuated valve assembly and the second luer-actuated valve assembly provides for opening of both the first and second seal elements and establishing of fluid flow through said fluid flow path and wherein disconnection of the first luer-actuated valve assembly and the second luer-actuated valve assembly provides for automatic closing of both the first and second sealing elements.

Further preferably, when the first and second luer-actuated valve assemblies are disconnected, the shuttle element is disposed in a forward position, such that the first sealing element is closed and wherein when the first and second luer-actuated valve assemblies are connected, the second luer-actuated valve assembly urges displacement of the shuttle element relative to the cannula against the urge of the biasing element, thereby opening both the first and the second seal elements.

Still further preferably, the connector assembly is configured to assume a first and a second operative orientation: the first operative orientation in which the first and second luer-actuated valve assemblies are connected in a snap-fit manner and the cannula is configured to open both the first seal element and the second seal element and thereby establish fluid communication between the first and second luer actuated valve assemblies; and the second operative orientation in which the first and second luer-actuated valve assemblies are disconnected following exertion of axial tensile force on the first and second luer-actuated valve assemblies in two different axial directions, and the first seal element and the second seal element are biased to their closed position, thereby preventing fluid communication between the first and second luer actuated valve assemblies.

Yet further preferably, the cannula has a conical forward end. Alternatively, the cannula has a cylindrical forward end.

In accordance with an embodiment of the present invention, the medical tube is selected from the group consisting of a catheter, an IV line and a drain. Preferably, each of the first seal element and the second seal element has a first open end and a second selectably openable end, including a selectively openable slit and wherein the first seal element and the second seal element is biased to its normally closed position when no compression force is exerted thereon.

Further preferably, tensile force in the range of 0.5 kgf-3 kgf is required in order to disconnect the first luer-actuated valve assembly from the second luer-actuated valve assembly. Still further preferably, in the second operative orientation the first and second luer-actuated valve assemblies are swabbable. Yet further preferably, the first luer-actuated valve assembly comprises a first housing which is integrally made or fixedly connected to the cannula, the shuttle being slidably seated within the first housing and the first seal element is retained within the shuttle by a male seal lock, which is fixedly coupled with the shuttle, such that in a closed position of the first seal element, the first seal element provides for a fluid tight sealing relative to the first housing.

In accordance with an embodiment of the present invention, engagement of the shuttle with the first housing, limits forward axial displacement of the shuttle relative to the first housing. Preferably, the second luer-actuated valve assembly comprises a second housing.

Further preferably, the second housing is integrally made or fixedly connected to a female seal cover and forming an interior volume therebetween, and wherein the second seal element is seated within the interior volume in a fluid tight sealing manner relative to the second housing and to the female seal cover. Still further preferably, the shuttle, the first seal element, the male seal lock are all displaceable axially forwardly together under the bias of the biasing element up to engagement of the shuttle with said first housing.

Yet further preferably, first coupling snaps are arranged on the first housing and second coupling snaps are arranged on the female seal cover, and wherein the first luer-actuated valve assembly and the second luer-actuated valve assembly are connected due to engagement of said first coupling snaps and the second coupling snaps. Preferably, during axial displacement of the first luer-actuated valve assembly and the second luer-actuated valve assembly in opposite axial directions, the first coupling snaps disengage the second coupling snaps, causing disconnection of the first luer-actuated valve assembly from the second luer-actuated valve assembly.

Alternatively, first coupling snaps are arranged on the first housing and second coupling snaps are arranged on the second housing, and wherein the first luer-actuated valve assembly and the second luer-actuated valve assembly are connected due to engagement of the first coupling snaps and the second coupling snaps. Preferably, during axial displacement of the first luer-actuated valve assembly and the second luer-actuated valve assembly in opposite axial directions, the first coupling snaps disengage the second coupling snaps, causing disconnection of the first luer-actuated valve assembly from the second luer-actuated valve assembly.

In accordance with an embodiment of the present invention, an automatically bidirectionally-sealable safe disconnect intra-medical tubing connector assembly comprising first and second luer-actuated valve assemblies configured for connection to opposite intermediate ends of a medical tube and configured to be connected to each other in a snap-fit manner and arranged for tensile force responsive disconnection; the first luer-actuated valve assembly comprises a first housing and a cannula defining a fluid flow path and a first seal element retained within a shuttle element, wherein the shuttle element is configured to be axially displaceable relative to the cannula; the second luer-actuated valve assembly comprises a second housing and a second seal element, and wherein the first housing and the second housing are configured to be connected to each other in a snap-fit manner.

Preferably, the shuttle element is configured to be axially displaceable relative to the cannula and further configured to be biased forwardly by a biasing element. Further preferably, connection of the first luer-actuated valve assembly and the second luer-actuated valve assembly provides for opening of both the first and second seal elements and establishing of fluid flow through the fluid flow path and wherein disconnection of the first luer-actuated valve assembly and the second luer-actuated valve assembly provides for automatic closing of both the first and second sealing elements.

Still further preferably, when the first and second luer-actuated valve assemblies are disconnected, the shuttle element is disposed in a forward position, such that the first sealing element is closed and wherein when the first and second luer-actuated valve assemblies are connected, the second luer-actuated valve assembly urges displacement of the shuttle element relative to the cannula against the urge of the biasing element, thereby opening both the first and the second seal elements.

Yet further preferably, the connector assembly is configured to assume a first and a second operative orientation: the first operative orientation in which the first and second luer-actuated valve assemblies are connected in a snap-fit manner and the cannula is configured to open both the first seal element and the second seal element and thereby establish fluid communication between the first and second luer actuated valve assemblies; and the second operative orientation in which the first and second luer-actuated valve assemblies are disconnected following exertion of axial tensile force on the first and second luer-actuated valve assemblies in two different axial directions, and the first seal element and the second seal element are biased to their closed position, thereby preventing fluid communication between the first and second luer actuated valve assemblies.

Preferably, the cannula has a conical forward end. Alternatively, the cannula has a cylindrical forward end.

Further preferably, the medical tube is selected from the group consisting of a catheter, an IV line and a drain.

In accordance with an embodiment of the present invention, each of the first seal element and said second seal element has a first open end and a second selectably openable end, including a selectively openable slit and wherein said first seal element and said second seal element is biased to its normally closed position when no compression force is exerted thereon.

Preferably, tensile force in the range of 0.5 kgf-3 kgf is required in order to disconnect the first luer-actuated valve assembly from the second luer-actuated valve assembly. Further preferably, in the second operative orientation the first and second luer-actuated valve assemblies are swabbable. Still further preferably, the shuttle being slidably seated within the first housing and the first seal element is retained within the shuttle by a male seal lock, which is fixedly coupled with the shuttle, such that in a closed position of the first seal element, the first seal element provides for a fluid tight sealing relative to the first housing.

Yet further preferably, engagement of the shuttle with the first housing, limits forward axial displacement of the shuttle relative to the first housing.

In accordance with an embodiment of the present invention, the second housing is integrally made or fixedly connected to a female seal cover and forming an interior volume therebetween, and wherein the second seal element is seated within the interior volume in a fluid tight sealing manner relative to the second housing and to the female seal cover. Preferably, the shuttle, the first seal element, the male seal lock are all displaceable axially forwardly together under the bias of the biasing element up to engagement of the shuttle with the first housing.

Further preferably, first coupling snaps are arranged on the first housing and second coupling snaps are arranged on the female seal cover, and wherein the first luer-actuated valve assembly and the second luer-actuated valve assembly are connected due to engagement of the first coupling snaps and the second coupling snaps. Still further preferably, during axial displacement of the first luer-actuated valve assembly and the second luer-actuated valve assembly in opposite axial directions, the first coupling snaps disengage the second coupling snaps, causing disconnection of the first luer-actuated valve assembly from the second luer-actuated valve assembly.

Alternatively, first coupling snaps are arranged on the first housing and second coupling snaps are arranged on the second housing, and wherein the first luer-actuated valve assembly and the second luer-actuated valve assembly are connected due to engagement of the first coupling snaps and the second coupling snaps. Preferably, during axial displacement of the first luer-actuated valve assembly and the second luer-actuated valve assembly in opposite axial directions, the first coupling snaps disengage the second coupling snaps, causing disconnection of the first luer-actuated valve assembly from the second luer-actuated valve assembly.

### BRIEF DESCRIPTION OF THE DRAWINGS

The present invention will be understood and appreciated more fully from the following detailed description, taken in conjunction with the drawings in which:

FIGS. 1A and 1B are respectively a simplified exploded illustration and a sectional exploded illustration of a safe disconnect intra-medical tubing connector assembly having a first valve assembly and a second valve assembly, arranged and constructed in accordance with an embodiment of the present invention, section being taken along lines B-B in FIG. 1A;

FIGS. 2A-2D are simplified respective perspective, planar top view, planar side view and a sectional illustration taken along lines D-D in FIG. 2C of a male connector, forming part of the first valve assembly of the safe disconnect intra-medical tubing connector assembly of FIGS. 1A and 1B;

FIGS. 3A-3E are simplified respective two perspective view shown from different directions, planar side view, planar top view and a sectional illustration taken along lines E-E in FIG. 3D of a shuttle, forming part of the first valve assembly of the safe disconnect intra-medical tubing connector assembly of FIGS. 1A and 1B;

FIGS. 4A-4C are simplified respective perspective, planar side view and sectional illustration taken along lines C-C in FIG. 4B of a male seal element, forming part of the first valve assembly of the safe disconnect intra-medical tubing connector assembly of FIGS. 1A and 1B;

FIGS. 5A-5C are simplified respective perspective, planar side view and sectional illustration taken along lines C-C in FIG. 5B of a male seal lock, forming part of the first valve assembly of the safe disconnect intra-medical tubing connector assembly of FIGS. 1A and 1B;

FIGS. 6A-6F are simplified respective perspective, two different planar side views, planar top view and two sectional illustrations taken along lines E-E in FIG. 6B and lines F-F in FIG. 6C of a male connector housing, forming part of the first valve assembly of the safe disconnect intra-medical tubing connector assembly of FIGS. 1A and 1B;

FIGS. 7A-7C are simplified respective perspective, planar side view and sectional illustration taken along lines C-C in FIG. 7B of a female seal cover, forming part of the second valve assembly of the safe disconnect intra-medical tubing connector assembly of FIGS. 1A and 1B;

FIGS. 8A-8C are simplified respective perspective, planar top view and sectional illustration taken along lines C-C in FIG. 8B of a female seal element, forming part of the second valve assembly of the safe disconnect intra-medical tubing connector assembly of FIGS. 1A and 1B;

FIGS. 9A-9C are simplified respective perspective, planar side view and a sectional illustration taken along lines C-C in FIG. 9B of a female connector, forming part of the second valve assembly of the safe disconnect intra-medical tubing connector assembly of FIGS. 1A and 1B;

FIGS. 10A and 10B are simplified respective perspective view and a sectional illustration taken along lines B-B in FIG. 10A of the safe disconnect intra-medical tubing connector assembly of FIGS. 1A and 1B, shown in a disconnected operative orientation, when the first valve assembly and the second valve assembly are disconnected;

FIGS. 11A and 11B are simplified respective planar side view and a sectional illustration taken along lines B-B in FIG. 11A of the safe disconnect intra-medical tubing connector assembly of FIGS. 1A and 1B, shown in a connected operative orientation, when the first valve assembly and the second valve assembly are connected.

FIGS. 12A and 12B are respectively a simplified exploded illustration and a sectional exploded illustration of a breakaway intra-medical tubing connector assembly having a first valve assembly and a second valve assembly, arranged and constructed in accordance with another embodiment of the present invention, section being taken along lines B-B in FIG. 12A;

FIGS. 13A-13C are simplified respective perspective, planar side view and a sectional illustration taken along lines C-C in FIG. 13B of a male connector, forming part of the first valve assembly of the breakaway intra-medical tubing connector assembly of FIGS. 12A and 12B;

FIGS. 14A-14C are simplified respective perspective, planar top view and a sectional illustration taken along lines C-C in FIG. 14B of a shuttle, forming part of the first valve assembly of the breakaway intra-medical tubing connector assembly of FIGS. 12A and 12B;

FIGS. 15A-15D are simplified respective perspective, planar side view, planar top view and sectional illustration taken along lines D-D in FIG. 15C of a male seal element, forming part of the first valve assembly of the breakaway intra-medical tubing connector assembly of FIGS. 12A and 12B;

FIGS. 16A-16D are simplified respective perspective, planar side view, planar top view and sectional illustration taken along lines D-D in FIG. 16C of a male seal lock, forming part of the first valve assembly of the breakaway intra-medical tubing connector assembly of FIGS. 12A and 12B;

FIGS. 17A-17D are simplified respective perspective, planar side view, planar top view and sectional illustration taken along lines D-D in FIG. 17C of a stopping ring, forming part of the first valve assembly of the breakaway intra-medical tubing connector assembly of FIGS. 12A and 12B;

FIGS. 18A-18D are simplified respective perspective, planar side view, planar top view and sectional illustration taken along lines D-D in FIG. 18C of a female seal cover, forming part of the second valve assembly of the breakaway intra-medical tubing connector assembly of FIGS. 12A and 12B;

FIGS. 19A-19C are simplified respective perspective, planar side view and sectional illustration taken along lines C-C in FIG. 19B of a female seal element, forming part of the second valve assembly of the breakaway intra-medical tubing connector assembly of FIGS. 12A and 12B;

FIGS. 20A-20D are simplified respective perspective, planar side view, planar top view and a sectional illustration taken along lines D-D in FIG. 20C of a female connector, forming part of the second valve assembly of the breakaway intra-medical tubing connector assembly of FIGS. 12A and 12B;

FIGS. 21A-21D are simplified respective perspective, two different planar side views and a sectional illustration taken along lines D-D in FIG. 21C of the breakaway intra-medical tubing connector assembly of FIGS. 12A and 12B, shown in a disconnected operative orientation, when the first valve assembly and the second valve assembly are disconnected;

FIGS. 22A-22D are simplified respective two perspective views taken from opposite directions, a planar side view and a sectional illustration taken along lines D-D in FIG. 22C of the breakaway intra-medical tubing connector assembly of FIGS. 12A and 12B, shown in a connected operative orientation, when the first valve assembly and the second valve assembly are connected.

### DESCRIPTION OF EMBODIMENTS OF THE INVENTION

An automatically bidirectionally-sealable safe disconnect intra-medical tubing connector assembly is provided in accordance with an embodiment of the present invention. The safe disconnect intra-medical tubing connector assembly preferably consists of a first and a second luer-actuated valve assemblies, which are configured to be selectably connected to each other to permit fluid flow passage therethrough. The first and second luer-actuated valve assemblies are configured to be disconnected from each other upon exertion of a certain tensile force, thereby causing automatic closing of both the first and second luer-actuated valves.

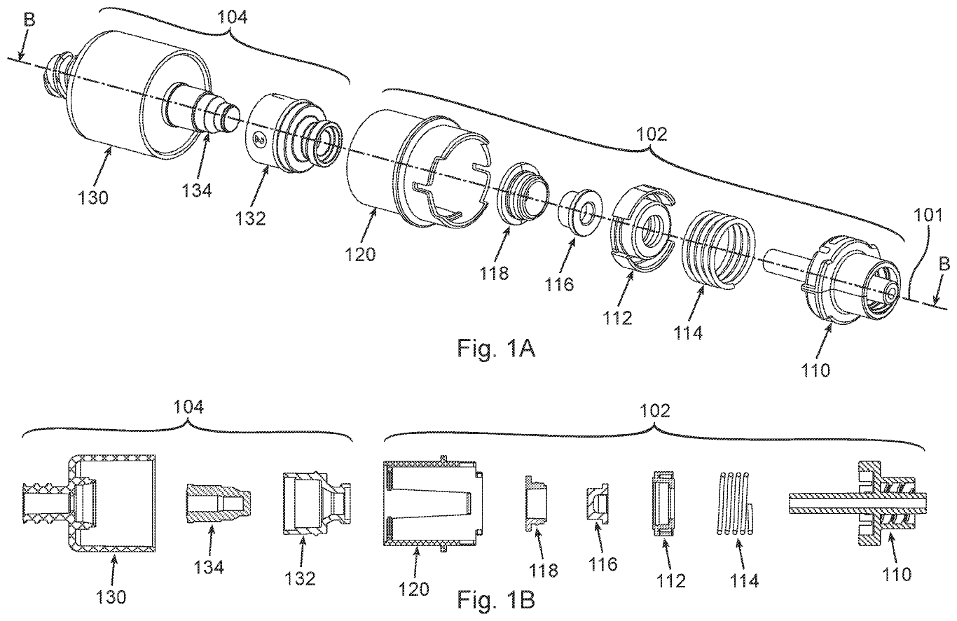

Reference is now made to FIGS. 1A and 1B, which are respectively a simplified exploded illustration and a sectional exploded illustration of a safe disconnect intra-medical tubing connector assembly having a first valve assembly and a second valve assembly, arranged and constructed in accordance with an embodiment of the present invention, section being taken along lines B-B in FIG. 1A.

A safe disconnect intra-medical tubing connector assembly 100 arranged along a longitudinal axis 101 is seen in FIGS. 1A & 1B. The safe disconnect intra-medical tubing connector assembly 100 preferably includes a first luer-actuated valve assembly 102 and a second luer-actuated valve assembly 104.

The first luer-actuated valve assembly 102 preferably includes a male connector 110, a shuttle 112 preferably seated within the male connector 110 and configured to be axially moveable relative thereto and further configured to be biased forwardly by a spring 114. A male seal element 116 is at least partially seated within the shuttle 112 and held therewithin by a male seal lock 118, which is fixedly coupled with the shuttle 112, such that the male seal element 116 provides for a fluid tight sealing relative to the male connector 110. A male connector housing 120 is preferably fixedly attached to the male connector 110 and configured to generally surround the shuttle 112, spring 114, male seal element 116 and male seal lock 118 and limit forward axial displacement of the shuttle 112 relative to the male connector 110.

It is noted that the male connector 110 and the male connector housing 120 can be either integrally formed or fixedly connected to each other.

The second luer-actuated valve assembly 104 preferably includes a female connector 130, a female seal cover 132 fixedly coupled thereto and forming an interior volume therebetween. A female seal element 134 is preferably seated within the interior volume in a fluid tight sealing manner relative to the female connector 130 and the female seal cover 132.

It is a particular feature of an embodiment of the present invention that the first luer-actuated valve assembly 102 is selectably connectable with the second luer-actuated valve assembly 104 in a tensile force responsive disconnectable snap fit connection manner.

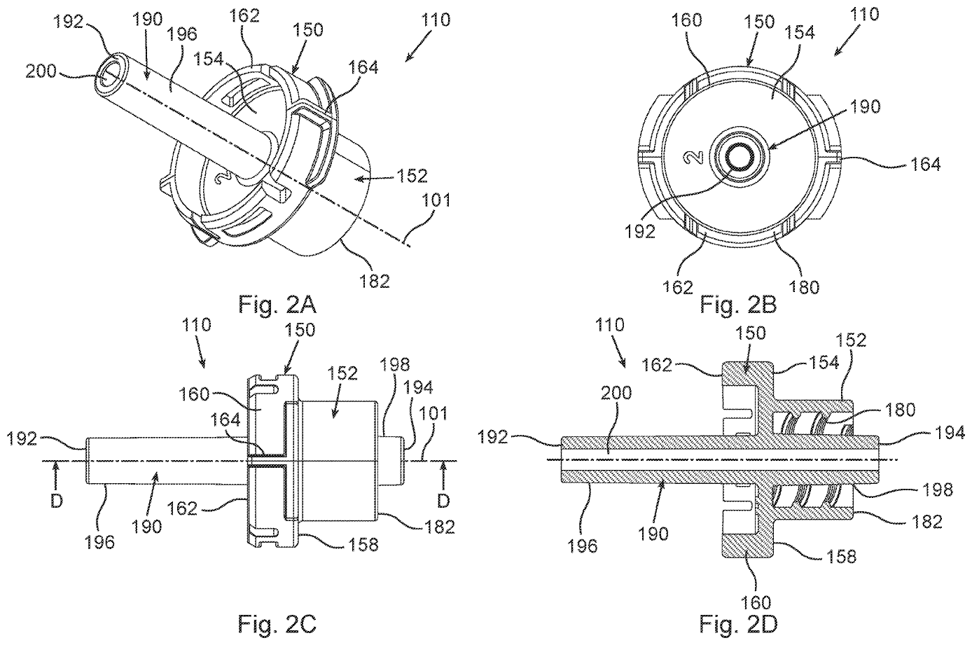

Reference is now made to FIGS. 2A-2D , which are simplified respective perspective, planar top view, planar side view and a sectional illustration taken along lines D-D in FIG. 2C of the male connector 110, forming part of the first valve assembly 102 of the safe disconnect intra-medical tubing connector assembly 100 of FIGS. 1A and 1B.

The male connector 110 is preferably an integrally molded element arranged along the longitudinal axis 101 and having a generally annular forward portion 150 and a generally cylindrical rearward portion 152 divided by a circular flange 154. A rearwardly facing annular shoulder 158 is formed on the circular flange 154. The rearward portion 152 has an inner threading 180 and extends from the rearwardly facing annular shoulder 158 to a rearwardmost circumferential end 182.

It is specifically seen in FIG. 2D that a cannula 190 extends coaxially within both the forward portion 150 and the rearward portion 152. The cannula 190 protrudes axially forwardly from the circular flange 154 and terminates at a forward edge 192. The cannula 190 also has a rearward edge 194, which is disposed slightly rearwardly from the rearwardmost circumferential end 182 of the rearward portion 152. The cannula 190 has a forward cannula portion 196 which extends forwardly from the circular flange 154 and a rearward cannula portion 198, which extends rearwardly from the circular flange 154, slightly protrudes rearwardly from the rearward portion 152 and is partially encircled by the inner threading 180. Both the forward cannula portion 196 and the rearward cannula portion 198 is preferably slightly conical, compliant with the ISO luer standards. Alternatively, both the forward cannula portion 196 and the rearward cannula portion 198 is preferably cylindrical. A fluid flow path 200 is formed through the cannula 190.

The forward portion 150 has a generally annular wall 160 extending forwardly from the circular flange 154 and terminating at a forwardmost edge 162. Typically, two diametrically opposed ribs 164 are formed on an outer surface of the annular wall 160.

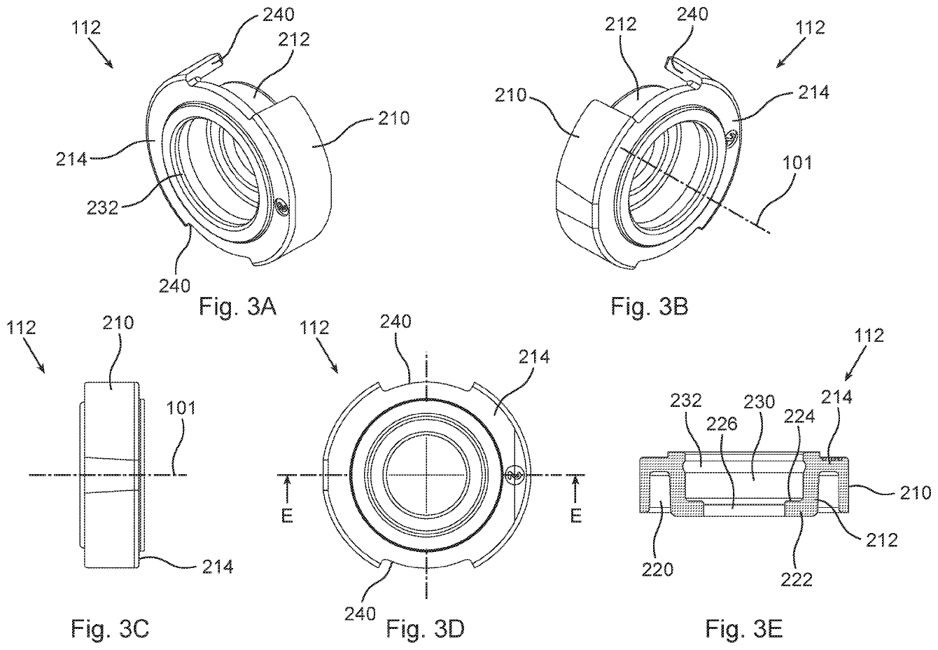

Reference is now made to FIGS. 3A-3E , which are simplified respective two perspective views shown from different directions, planar side view, planar top view and a sectional illustration taken along lines E-E in FIG. 3D of the shuttle 112, forming part of the first valve assembly 102 of the safe disconnect intra-medical tubing connector assembly 100 of FIGS. 1A and 1B.

The shuttle 112 is preferably an integrally molded disc-shaped element arranged about the longitudinal axis 101. The shuttle 112 typically has two radially spaced concentrical annular walls 210 and 212, an outer annular wall 210 and an inner annular wall 212 connected by a forward circular flange 214, which extends transversely with respect to the annular walls 210 and 212. An annular recess 220 is formed between the walls 210 and 212.

An annular flange 222 extends radially inwardly from a rearward end of the inner annular wall 212 and defines a forwardly facing shoulder 224. A bore 226 is formed through the annular flange 222.

It is further seen in FIG. 3E that a socket 230 is formed in the shuttle 112 and defined by the inner annular wall 212 and the annular flange 222. Preferably, a circumferential groove 232 is formed on the inner surface of the inner annular wall 212 and disposed slightly rearwardly from the circular flange 214.

Typically, two diametrically opposed cut-outs 240 are provided on the annular wall 210.

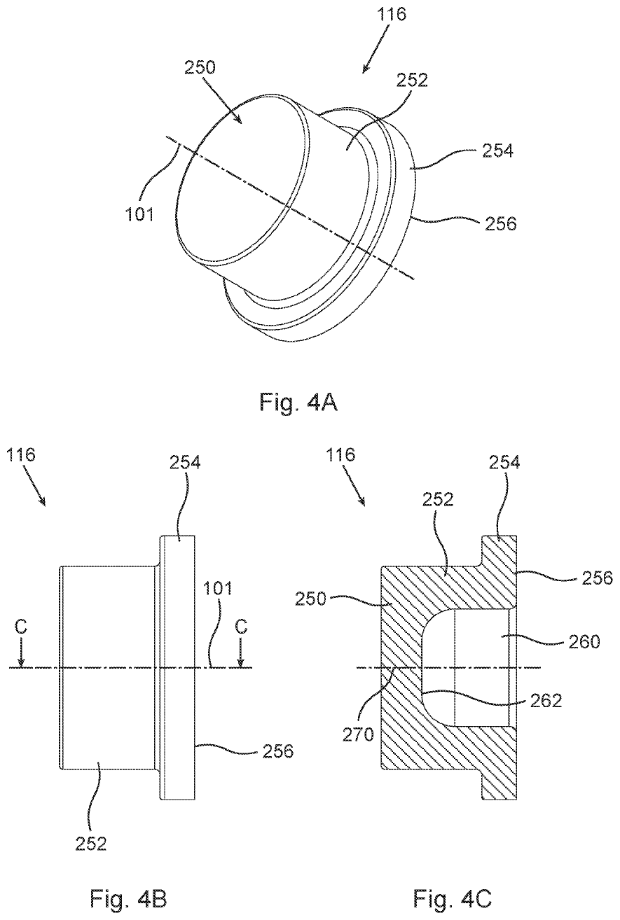

Reference is now made to FIGS. 4A-4C , which are simplified respective perspective, planar side view and sectional illustration taken along lines C-C in FIG. 4B of the male seal element 116, forming part of the first valve assembly 102 of the safe disconnect intra-medical tubing connector assembly 100 of FIGS. 1A and 1B.

The male seal element 116 is arranged about longitudinal axis 101 and is preferably integrally molded, and preferably made of plastic, such as TPE, Polyethylene or any other suitable resilient plastic material.

It is seen in FIGS. 4A-4C that the male seal element 116 is generally cup-shaped and has a generally circular sealing portion 250 and a generally cylindrical portion 252 extending rearwardly therefrom, transversely with respect thereto. An annular flange 254 extends radially outwardly from the rearward end of the cylindrical portion 252 and defines a rearwardmost annular edge 256. Optionally, a circular groove may be formed on the outer surface of the cylindrical portion 252 to serve as the fulcrum point of the male seal element 116 ensuring its elastic deformation upon exertion of compressive pressure thereon.

A socket 260 is formed within the male seal element 116 and extends from the rearwardmost annular edge 256 to a rearwardly facing surface 262.

A normally closed slit 270 is formed through the circular sealing portion 250.

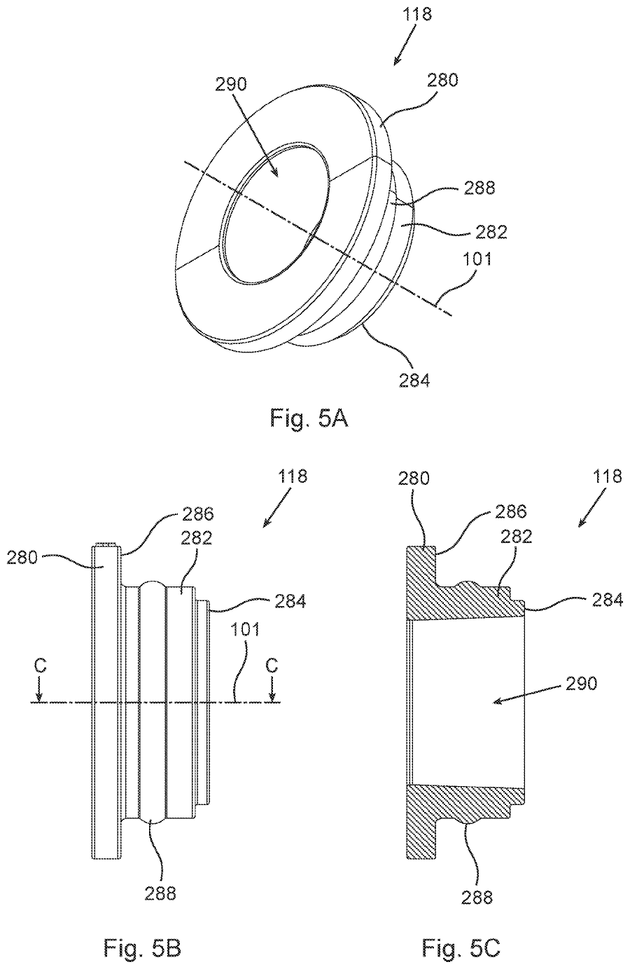

Reference is now made to FIGS. 5A-5C , which are simplified respective perspective, planar side view and sectional illustration taken along lines C-C in FIG. 5B of the male seal lock 118, forming part of the first valve assembly 102 of the safe disconnect intra-medical tubing connector assembly 100 of FIGS. 1A and 1B.

The male seal lock 118 is arranged about the longitudinal axis 101 and is preferably an integrally molded element.

It is seen in FIGS. 5A-5C that the male seal element 118 has a generally annular flange portion 280 and a generally cylindrical portion 282 extending rearwardly therefrom, transversely with respect thereto and terminating at a rearwardmost annular edge 284. The annular flange portion 280 defines a rearwardly facing shoulder 286 extending radially outwardly with respect to the cylindrical portion 282.

An annular protrusion 288 is formed on the outer surface of the cylindrical portion 282 and preferably disposed at an intermediate location along the longitudinal extent thereof. A bore 290 is formed through the male seal element 118.

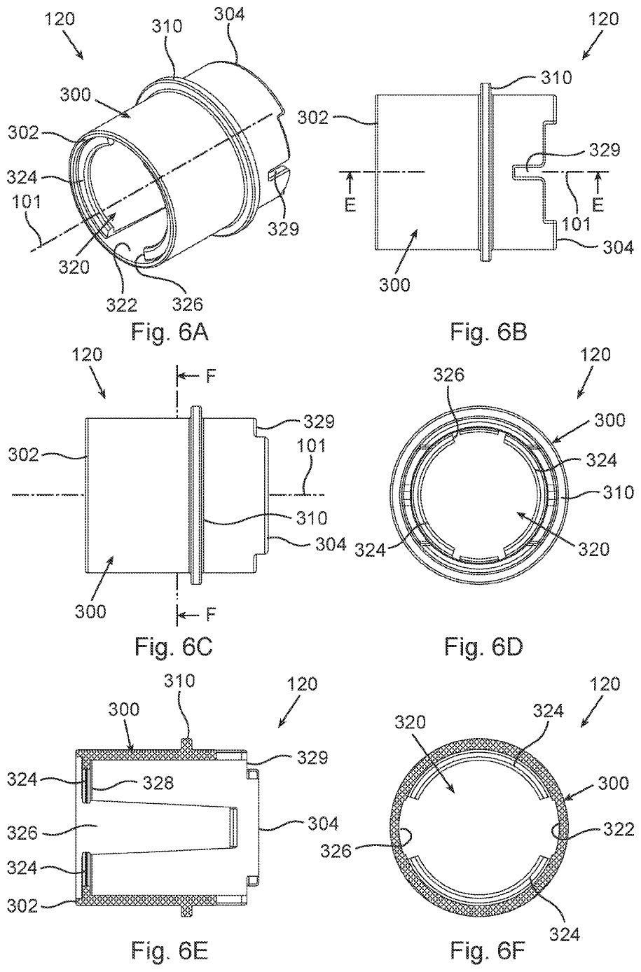

Reference is now made to FIGS. 6A-6F , which are simplified respective perspective, two different planar side views, planar top view and two sectional illustrations taken along lines E-E in FIG. 6B and lines F-F in FIG. 6C of the male connector housing 120, forming part of the first valve assembly 102 of the safe disconnect intra-medical tubing connector assembly 102 of FIGS. 1A and 1B.

The male connector housing 120 is arranged about the longitudinal axis 101 and is preferably an integrally molded element.

It is seen in FIGS. 6A-6E that the male connector housing 120 has a generally cylindrical body 300, defining a forwardmost edge 302 and a rearwardmost edge 304.

It is noted that a generally circumferential flange 310 is formed on an outer surface of the body 300 of the male connector housing 120 and is located generally adjacent the rearwardmost edge 302. The flange 310 extends radially outwardly from the outer surface of the body 300 and is generally annular. Alternatively, the flange 310 can be comprised of several circumferential segments, rather than having a continuous annular edge. A generally cylindrical troughing bore 320 is formed within the body 300 and is defined by an inner surface 322 of the body 300.

It is a particular feature of an embodiment of the present invention that typically, two partially circular coupling snaps 324 extend radially inwardly from the inner surface 322 of the body 300 and are disposed slightly rearwardly of the forwardmost edge 302. Gaps 326 are preferably provided between the coupling snaps 324. Alternatively, a single continuous annular radially inwardly directed coupling snap could have been provided instead of the partially circular coupling snaps 324.

The coupling snaps 324 each defines a rearwardly facing shoulder 328.

Typically, two diametrically opposed cut-outs 329 are formed adjacent the rearward most end 304 of the body 300.

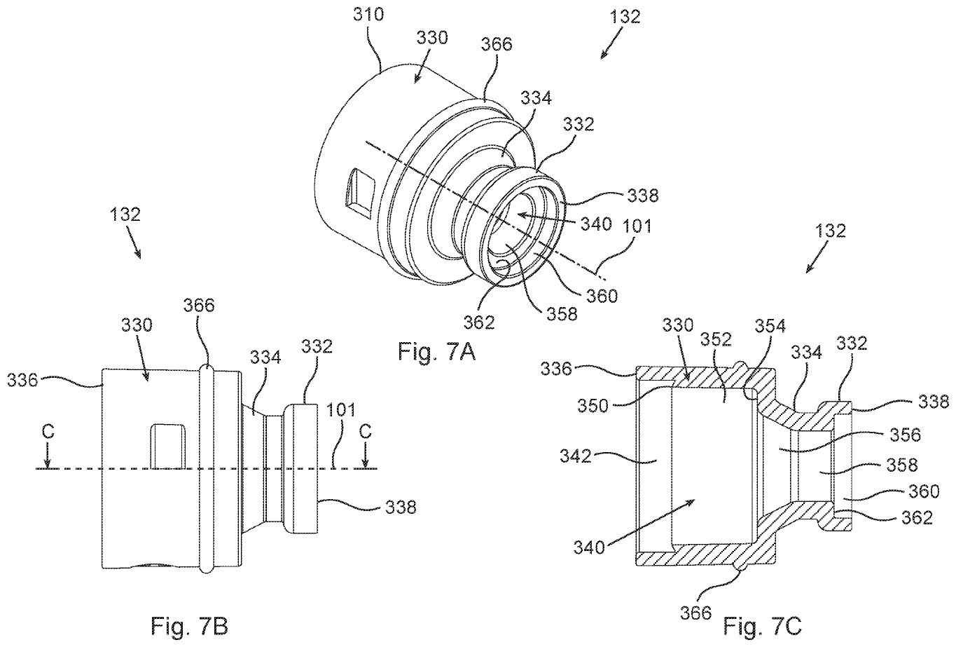

Reference is now made to FIGS. 7A-7C , which are simplified respective perspective, planar side view and sectional illustration taken along lines C-C in FIG. 7B of the female seal cover 132, forming part of the second valve assembly 104 of the safe disconnect intra-medical tubing connector assembly 100 of FIGS. 1A and 1B.

The female seal cover 132 is arranged along the longitudinal axis 101 and is preferably an integrally molded element. The female seal element 132 preferably includes a forward cylindrical portion 330 and a rearward neck portion 332 disposed rearwardly therefrom and having a generally smaller outer diameter with respect to the forward cylindrical portion 330. A rearwardly tapered portion 334 joins the forward cylindrical portion 330 with the rearward neck portion 332. The forward cylindrical portion 330 defines a forwardmost circumferential edge 336 and the rearward neck portion 332 defines a forwardmost circumferential edge 338.

A through bore 340 is formed through the female seal element 132 and typically comprises a forward portion 342 extending from the forwardmost circumferential edge 336 to a forwardly facing shoulder 350, an intermediate portion 352 extending from the forwardly facing shoulder 350 to a forwardly facing surface 354 disposed adjacent the rearwardly tapered portion 334. The through bore 340 further comprises a tapered rearward portion 356, a rearward portion 358 and a rearwardmost portion 360 terminating adjacent the forwardmost circumferential edge 338. The rearwardmost portion 360 defines a rearwardly facing shoulder 362 disposed slightly forwardly of the forwardmost circumferential edge 338.

It is a particular feature of an embodiment of the present invention that a generally circumferential coupling snap 366 is formed on an outer surface of the cylindrical portion 330 of the female seal cover 132 and is located generally adjacent the tapered portion 334. The coupling snap 366 extends radially outwardly from the outer surface of the cylindrical portion 330 and is generally annular. Alternatively, the coupling snap 366 can be comprised of several circumferential segments, rather than having a continuous annular edge.

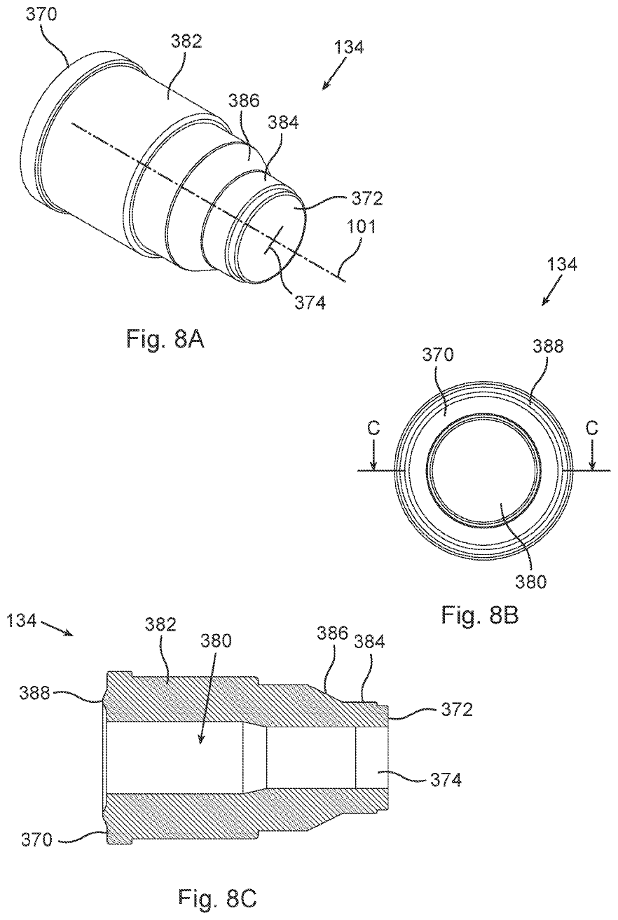

Reference is now made to FIGS. 8A-8C , which are simplified respective perspective, planar side view and sectional illustration taken along lines C-C in FIG. 8B of the female seal element 134, forming part of the second valve assembly 104 of the safe disconnect intra-medical tubing connector assembly 100 of FIGS. 1A and 1B.

It is appreciated that the female seal element 134 is essentially similar to various commercially available seal elements from various manufacturers, such as Haemopharm, Halkey-Roberts, NP Medical.

The female seal element 134 is preferably resilient and compressible and has a forward open end 370 and a rearward selectably openable end 372, including a selectively openable slit 374. The rearward selectably openable end 372 of the female seal element 134 is disposed in its normally closed operative orientation at rest, when the female seal element 134 is not compressed. It is noted that the female seal element 134 is biased to its normally closed operative orientation when no pressure is applied thereupon. A fluid flow passage 380 is defined by the inner volume of the female seal element 134. The rearward selectably openable end 372 provides a swabbable surface that may be cleaned by the physician in order to prevent contamination. The female seal element 134 includes a generally cylindrical portion 382 adjacent the forward open end 370 thereof, a rearward generally cylindrical portion 384 adjacent the rearward selectably openable end 372, having a diameter that is generally smaller with respect to the forward cylindrical portion 382 and a tapered portion 386 joining the two cylindrical portions 382 and 384.

A generally annular protrusion 388 extends generally forwardly from the forward open end 370 and generally surrounds the fluid flow passage 380.

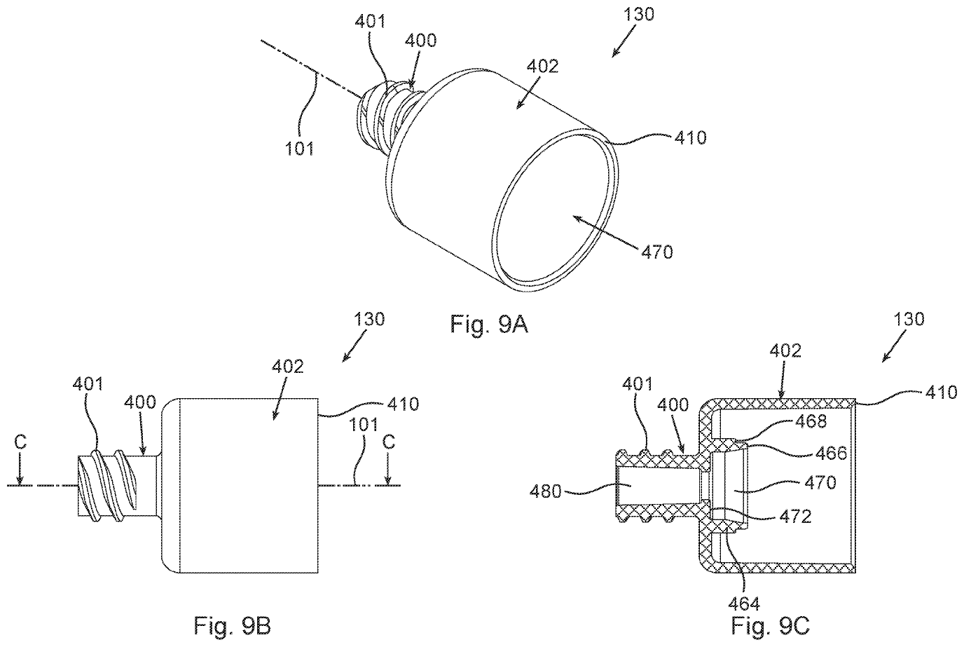

Reference is now made to FIGS. 9A-9C , which are simplified respective perspective, planar side view and a sectional illustration taken along lines C-C in FIG. 9B of the female connector 130, forming part of the second valve assembly 104 of the safe disconnect intra-medical tubing connector assembly 100 of FIGS. 1A and 1B.

The female connector 130 has a female luer portion 400 with an external threading 401 and a hollow cup-shaped rearward connector portion 402 extending rearwardly therefrom to a rearwardmost end surface 410.

A cup-shaped protrusion 464 is arranged co-axially with the connector portion 402 and extends rearwardly from the forward end of the connector portion 402 to a circumferential rearward end surface 466. A rearwardly facing shoulder 468 is formed on the outer surface of the protrusion 464 and is slightly forwardly spaced from the rearward end surface 466. The cup-shaped protrusion 464 has a socket 470 defining a rearwardly facing surface 472 and fluidly communicating with a bore 480 formed through the female luer portion 400.

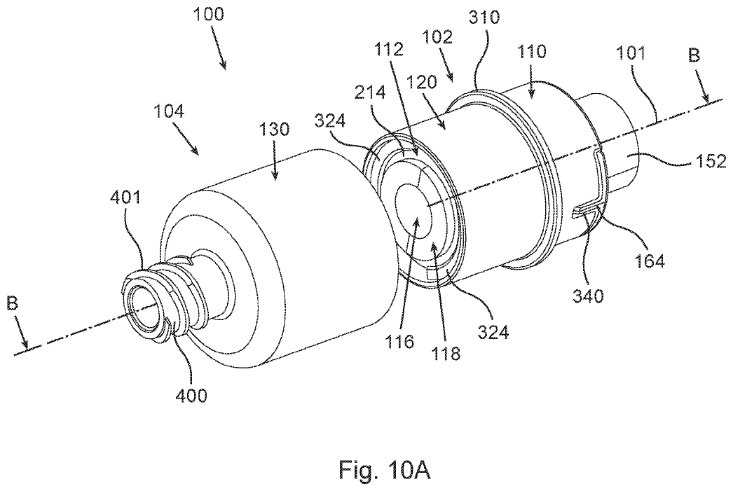

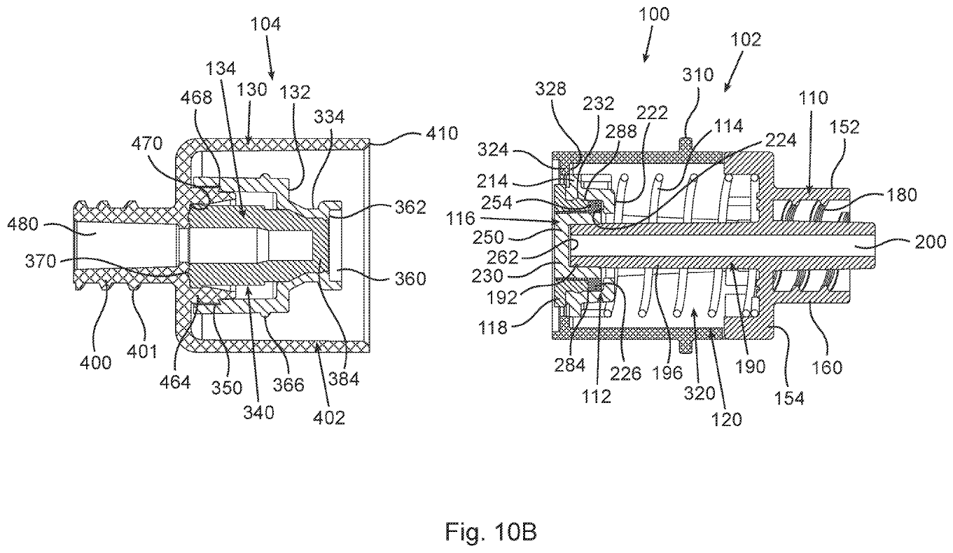

Reference is now made to FIGS. 10A and 10B, which are simplified respective perspective view and a sectional illustration taken along lines B-B in FIG. 10A of the safe disconnect intra-medical tubing connector assembly 100 of FIGS. 1A and 1B, shown in a disconnected operative orientation, when the first valve assembly 102 and the second valve assembly 104 are disconnected.

It is seen in FIGS. 10A & 10B that the first valve assembly 102 and the second valve assembly 104 are disconnected from each other and their respective seal elements, namely the male seal element 116 and the female seal element 134 are sealingly disposed in their normally closed operative orientation.

It is specifically seen in FIG. 10B that the male seal element 116 is fixedly retained between the shuttle 112 and the male seal lock 118. It is further seen that a spring 114 is supported between the male connector 110 and the shuttle 112 and thereby biases the shuttle along with the male seal element 116 and the male seal lock 118 forwardly up to engagement of the shuttle 112 with coupling snaps 324 of the male connector housing 120.

It is noted that the shuttle 112 with the male seal element 116 and the male seal lock 118 biased by the spring 114 are all disposed within the througoing bore 320 of the male connector housing 120 and retained therewithin by the coupling snaps 324 thereof. Particularly, the forward circular flange 214 of the shuttle 112 engages rearwardly facing shoulder 328 of coupling snaps 324 of the male connector housing 120, thereby the sub-assembly comprised of the the male seal lock 118, the male seal element 116 and the shuttle 112 is prevented from axial forward displacement under the urge of the spring 114.

It is a particular feature of an embodiment of the present invention that the male seal element 116 engages the forward edge 192 of the forward cannula portion 196 of the male connector 110 in this disconnected operative orientation, such that the sealing portion 250 of the male seal element 116 seats across the fluid flow path 200 of the cannula 190 of the male connector 110 with the slit 270 closed and thus seals the fluid flow passage therethrough.

The sealing portion 250 of the male seal element 116 provides a swabbable surface that may be cleaned by the physician in order to prevent contamination.

It is noted that the male seal element 116 is normally closed at rest, unless penetrated by the cannula 190 of the male connector 110.

It is a particular feature of an embodiment of the present invention that the shuttle 112 along with the male seal element 116 is axially moveable relative to the male connector 110 and thus relative to the cannula 190 of the male connector 110 and is biased forwardly under the urge of the spring 114. In this disconnected operative orientation, the shuttle 112 is disposed in its forwardmost position and its further forward axial displacement is prevented by engagement with the coupling snaps 324 of the male connector housing 120. Specifically, the circular flange 214 of the shuttle 112 engages the rearwardly facing shoulders 328 of the coupling snaps 324 of the male connector housing 120 in this forward position of the shuttle 112.

It is particularly seen in FIG. 10B that the spring 114 is supported between the circular flange 154 of the male connector 110 and the circular flange 214 of the shuttle 112.

It is further seen in FIG. 10B that the male seal element 116 is seated between the shuttle 112 and the male seal element 118 such that the annular flange 254 of the male seal element is supported between the forwardly facing shoulder 224 of the shuttle 112 and the rearwardmost annular edge 284 of the male seal lock 118. The male seal lock 118 is fixedly attached to the shuttle 112 by means of engagement of the annular protrusion 288 of the male seal lock 118 with the circumferential groove 232 of the shuttle 112. Thus, the shuttle 112, the male seal element 116 and the male seal lock 118 are axially displaceable together as one unit.

It is further seen in FIG. 10B that the female seal element 134 is fixedly retained between the female connector 130 and the female seal cover 132, whereas the female seal cover 132 is fixedly attached to the female connector 130 by means of engagement between the forwardly facing shoulder 350 of the female seal cover 132 and the rearwardly facing shoulder 468 of the female connector 130.

The female seal element 134 is seated partially within the socket 470 of the cup-shaped protrusion 464 of the female connector 130 and partially within the trough bore 340 of the female seal cover 132. It is specifically seen that the cylindrical portion 384 of the female seal element 134 is seated within the rearward portion 358 of the trough bore 340 of the female seal cover 132 and is disposed in its normally closed operative orientation such that slit 374 is sealed and fluid flow passage into bore 480 is prevented.

It is noted that the female seal element 134 is biased to its normally closed position once a compression force exerted thereon is removed.

The female seal element 134 is supported against the female connector 130 by engagement of the forward open end 370 of the female seal element 134 with the forward end of the connector portion 402 of the female connector 130.

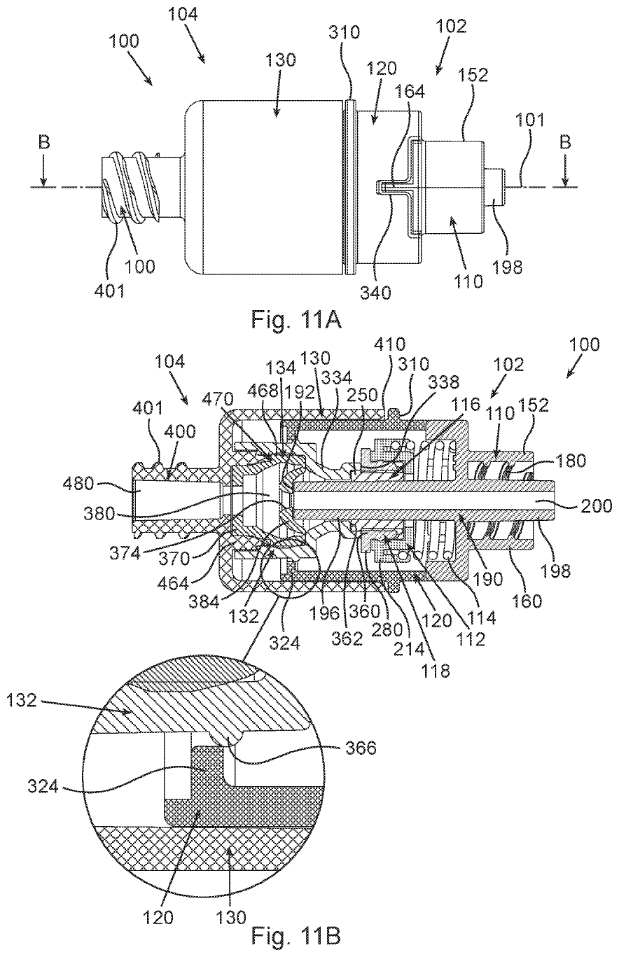

Reference is now made to FIGS. 11A and 11B, which are simplified respective planar side view and a sectional illustration taken along lines B-B in FIG. 11A of the safe disconnect intra-medical tubing connector assembly 100 of FIGS. 1A and 1B, shown in a connected operative orientation, when the first valve assembly 102 and the second valve assembly 104 are connected.

It is seen in FIGS. 11A & 11B that the first valve assembly 102 and the second valve assembly 104 are connected to each other and their respective seal elements, namely the male seal element 116 and the female seal element 134 are now open and allow fluid flow passage through the cannula 190 of the male connector 110 of the first valve assembly 102, via the opened slit 374 and the fluid flow passage 380 of the female seal element 134 and into bore 480 of the female connector 130 of the second valve assembly 104.

It is appreciated that all spatial relationships between the various components of the safe disconnect intra-medical tubing connector assembly 100 remain the same as described hereinabove with respect to the disconnected operative orientation illustrated in FIGS. 10A & 10B, besides the following spatial relationships:

It is a particular feature of an embodiment of the present invention, as particularly seen in FIGS. 11A & 11B, that upon connection of the first valve assembly 102 and the second valve assembly 104, the male connector housing 120 and the female seal cover 132 are selectably attached to each other in a tensile force responsive disconnectable snap-fit manner. Specifically, during axial displacement of the first valve assembly 102 and the second valve assembly 104 in opposite axial directions, the coupling snaps 324 of the male connector housing 120 engage the coupling snap 366 of the female seal cover 132 and causes momentary radial outward deflection of the coupling snaps 324 of the of the male connector housing 120, thus the coupling snaps 324 of the male connector housing 120 skip over the coupling snaps 366 of the female seal cover 132, causing disconnection of the first valve assembly 102 from the second valve assembly 104.

It is noted that the tensile force that is required in order to disconnect the first valve assembly 102 and the second valve assembly 104 is typically in the range of 0.5-3 kgf.

It is noted that flange 310 of the male connector housing 120 serves as a stopping feature upon connection of the first and second luer-actuated valve assemblies 102 and 104 and prevents further axial displacement of the first and second luer-actuated valve assemblies 102 and 104 axially towards each other.

It is a further particular feature of an embodiment of the present invention that in this connected operative orientation of the safe disconnect intra-medical tubing connector assembly 100, when the first valve assembly 102 and the second valve assembly 104 are attached to each other in a tensile force responsive disconnectable snap-fit manner, the cannula 190 of the male connector 110 causes opening of both the male seal element 116 and the female seal element 134 and thereby allows fluid flow passage through the first valve assembly 102 and the second valve assembly 104, specifically through the cannula 190 of the male connector 110 of the first valve assembly 102, via the opened slit 374 and the fluid flow passage 380 of the female seal element 134 and into bore 480 of the female connector 130 of the second valve assembly 104.

It is a still further particular feature of an embodiment of the present invention that upon connection of the first valve assembly 102 and the second valve assembly 104, a portion of the second valve assembly 104 urges rearward axial displacement of the shuttle 112 relative to the cannula 190 of the male connector 110 against the urge of the spring 114 and thereby causing opening of the male seal element 116 and the female seal element 134 by the cannula 190, permitting fluid flow passage from the first valve assembly 102 to the second valve assembly 104 or vice-versa.

As specifically seen in FIG. 11B, upon connection of the first valve assembly 102 and the second valve assembly 104 the forwardmost circumferential edge 338 of the female seal cover 132 engages the annular flange portion 280 of the male seal lock 118 and pushes the shuttle 112, along with the male seal element 116 and the male seal lock 118 axially rearwardly against the urge of the spring 114. It is seen in FIG. 11B that the spring 114 is now compressed and the forward end 196 of the cannula 190 of the male connector 110 penetrates the sealing portion 250 of the male seal element 116 through slit 270 and enters the rearward portion 358 of the through bore 340 of the female seal cover 132. Upon insertion of the forward end 196 of the cannula 190 of the male connector 110 into the through bore 340 of the male seal cover 132, the female seal element 134 is axially compressed and thus buckles and provides for opening of the slit 374. Once the cannula 190 of the male connector 110 penetrates the slit 270 of the male seal element 116 and causes opening of the slit 374 of the female seal element 134, fluid flow passage through the two valve assemblies 102 and 104 is established.

It is noted that the female luer portion 400 of the female connector 130 may be connected to a first medical tube and the rearward end 198 of the cannula 190 of the male connector 110 may be connected to a second medical tube, which may be adapted to be positioned in a certain treatment area within the patient's body, thus dis-location of one of the medical tubes from its desired position may require replacement of the entire medical set in absence of a connector such as the safe disconnect intra-medical tubing connector assembly 100 constructed and operative in accordance with an embodiment of the present invention.

It is noted that the fluid flow direction can be established in an opposite direction.

It is noted that compression forces exerted on both the male seal element 116 and the female seal element 134 by the cannula 190 are enabled by the fact that the first valve assembly 102 and the second valve assembly 104 are held in place relative to each other due to snap-fit connection between the male connector housing 120 and the female seal cover 132.

It is a particular feature of an embodiment of the present invention that upon application of tensile force in a direction of disconnection of the first valve assembly 102 and the second valve assembly 104, the coupling snap 324 of the male connector housing 120 skips over and disengages the coupling snap 366 of the female seal cover 132 and thus the cannula 190 of the male connector 110 is removed from the female seal cover 132, thereby causing sealing of the female seal element 134, due to the fact that it is biased to its normally closed position. Further, upon disengagement between the female seal cover 132 and the male seal lock 118, the spring 114 biases the shuttle 112, the male seal element 116 and the male seal lock 118 to move axially forwardly up to sealing of the slit 270 of the male seal element 116.

It is noted that the shuttle 112 is biased forwardly up to engagement thereof with the rearwardly facing shoulder 328 of the coupling snap 324 and upon disconnection of the first valve assembly 102 and the second valve assembly 104, the intra-medical tubing connector assembly 100 is positioned in its disconnected operative orientation again, as illustrated in FIGS. 10A & 10B, where no fluid flow passage is allowed through the valve assemblies 102 and 104.

It is a particular feature of an embodiment of the present invention that upon disconnection of the first and second valve assemblies 102 and 104, both of the respective seal elements 116 and 134 are automatically bidirectionally closed, thus preventing fluid flow passage from the two medical tubes.

It is noted that tensile force exerted on a medical set such as IV line may be unintentional, but due to the presence of the safe disconnect intra-medical tubing connector assembly 100, the catheter or other medical tube associated with at least one of the luer-actuated valve assemblies 102 and 104 remains in its position within the treatment site and the luer-actuated valve assemblies 102 and 104 are safely sealed once disconnected from each other. Therefore, no fluid can unintentionally flow out of the IV line and risk of contamination of the treatment site is prevented due to sealing of the luer-actuated valve assemblies 102 and 104 and the ability to clean the exterior surface of the sealing elements 116 and 134 thereof.

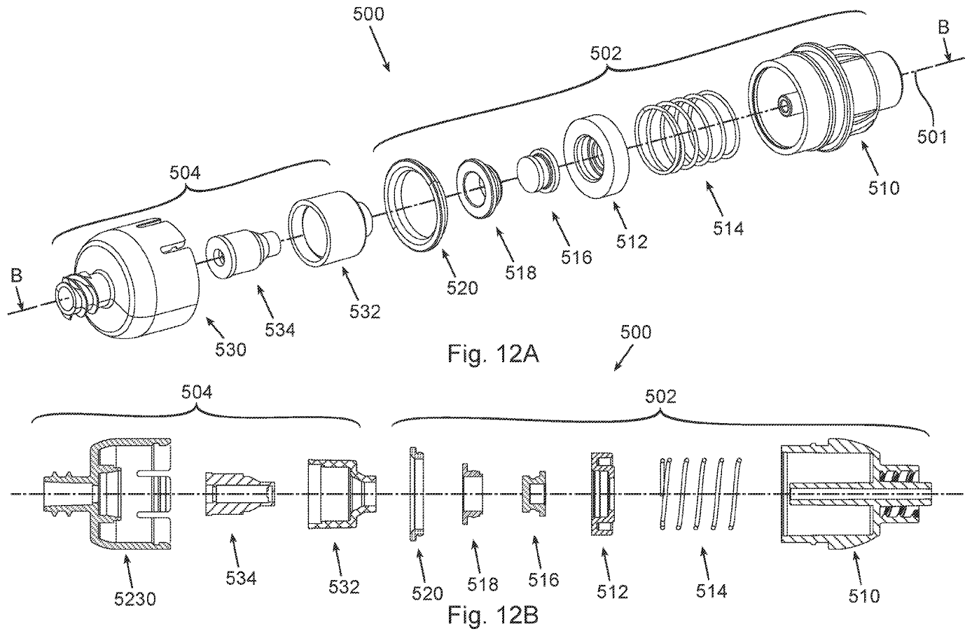

Reference is now made to FIGS. 12A and 12B, which are respectively a simplified exploded illustration and a sectional exploded illustration of a breakaway intra-medical tubing connector assembly having a first valve assembly and a second valve assembly, arranged and constructed in accordance with another embodiment of the present invention, section being taken along lines B-B in FIG. 12A.

A breakaway intra-medical tubing connector assembly 500 arranged along a longitudinal axis 501 is seen in FIGS. 12A & 12B. The breakaway intra-medical tubing connector assembly 500 preferably includes a first luer-actuated valve assembly 502 and a second luer-actuated valve assembly 504.

The first luer-actuated valve assembly 502 preferably includes a male connector 510, a shuttle 512 preferably seated within the male connector 510 and configured to be axially moveable relative thereto and further configured to be biased forwardly by a spring 514. A male seal element 516 is at least partially seated within the shuttle 512 and held therewithin by a male seal lock 518, which is fixedly coupled with the shuttle 512, such that the male seal element 516 provides for a fluid tight sealing relative to the male connector 510. A stopping ring 520 is preferably fixedly attached to the male connector 510 and configured to limit forward axial displacement of the shuttle 512 relative to the male connector 510.

The second luer-actuated valve assembly 504 preferably includes a female connector 530, a female seal cover 532 fixedly coupled thereto and forming an interior volume therebetween. A female seal element 534 is preferably seated within the interior volume in a fluid tight sealing manner relative to the female connector 530.

It is a particular feature of an embodiment of the present invention that the first luer-actuated valve assembly 502 is selectably connectable with the second luer-actuated valve assembly 504 in a tensile force responsive disconnectable snap fit connection manner.

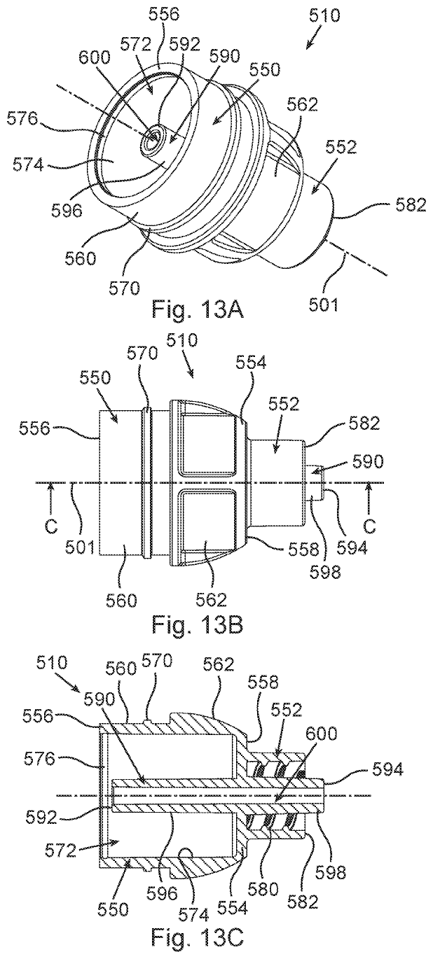

Reference is now made to FIGS. 13A-13C , which are simplified respective perspective, planar side view and a sectional illustration taken along lines C-C in FIG. 13B of the male connector 510, forming part of the first valve assembly 502 of the breakaway intra-medical tubing connector assembly 500 of FIGS. 12A and 12B.

The male connector 510 is preferably an integrally molded element arranged along the longitudinal axis 501 and having a generally cylindrical forward portion 550 and a generally cylindrical rearward portion 552 divided by a generally circular flange 554, which extends generally transversely with respect to longitudinal axis 501. The forward portion 550 extends from a forwardmost circumferential edge 556 to the circular flange 554, which defines a rearwardly facing annular shoulder 558.

The forward portion 550 has a forward attachment surface 560 extending rearwardly from the forwardmost circumferential edge 556 and a gripping surface 562, which is typically disposed adjacent the circular flange 554. A generally circumferential coupling snap 570 is formed on the forward attachment surface 560. It is seen in FIGS. 13A-13C that the coupling snap 570 is generally disposed at an intermediate location along the longitudinal extent of the forward attachment surface 560 and is rearwardly spaced from the forwardmost circumferential edge 556. The coupling snap 570 extends radially outwardly from the forward attachment surface 560 and is generally annular. Alternatively, the coupling snap 570 can be comprised of several circumferential segments, rather than having a continuous annular edge. A generally cylindrical socket 572 is formed within the forward portion 550 and is defined by an inner surface 574 of the forward portion 550 and the circular flange 554. A generally circumferential groove 576 is formed on the inner surface 574 and is slightly rearwardly spaced from the forwardmost circumferential edge 556.

The rearward portion 552 has an inner threading 580 and extends from the circular flange 554 to a rearwardmost circumferential end 582.

It is specifically seen in FIG. 13C that a cannula 590 extends coaxially within both the forward portion 550 and the rearward portion 552. The cannula 590 has a forward edge 592 disposed slightly rearwardly from the circumferential groove 576 and a rearward edge 594, which is disposed slightly rearwardly from the rearwardmost circumferential end 582 of the rearward portion 552. The cannula 590 has a forward cannula portion 596 which extends forwardly from the circular flange 554 and a rearward cannula portion 598, which extends rearwardly from the circular flange 554, slightly protrudes rearwardly from the rearward portion 552 and is partially encircled by the inner threading 580. Both the forward cannula portion 596 and the rearward cannula portion 598 are preferably slightly conical, compliant with the ISO luer standards. Alternatively, both the forward cannula portion 596 and the rearward cannula portion 598 are preferably cylindrical. A fluid flow path 600 is formed through the cannula 590.

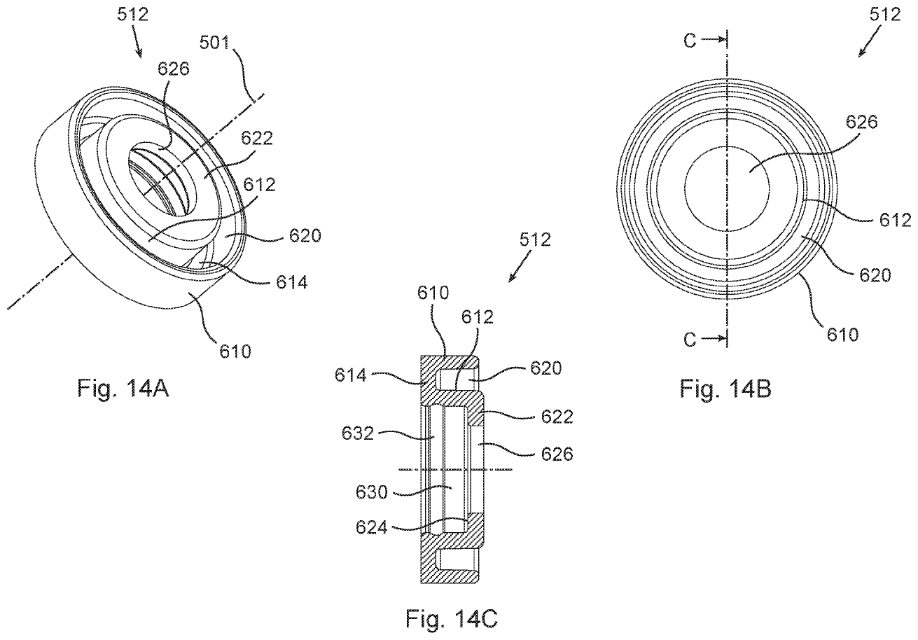

Reference is now made to FIGS. 14A-14C , which are simplified respective perspective, planar top view and a sectional illustration taken along lines C-C in FIG. 14B of the shuttle 512, forming part of the first valve assembly 502 of the breakaway intra-medical tubing connector assembly 500 of FIGS. 12A and 12B.

The shuttle 512 is preferably an integrally molded disc-shaped element arranged about the longitudinal axis 501. The shuttle 512 typically has two radially spaced concentrical annular walls 610 and 612, an outer annular wall 610 and an inner annular wall 612 connected by a forward circular flange 614, which extends transversely with respect to the annular walls 610 and 612. An annular recess 620 is formed between the walls 610 and 612.

An annular flange 622 extends radially inwardly from a rearward end of the inner annular wall 612 and defines a forwardly facing shoulder 624. A bore 626 is formed through the annular flange 622.

It is further seen in FIG. 14C that a socket 630 is formed in the shuttle 512 and defined by the inner annular wall 612 and the annular flange 622. Preferably, a circumferential groove 632 is formed on the inner surface of the inner annular wall 612 and disposed slightly rearwardly from the circular flange 614.

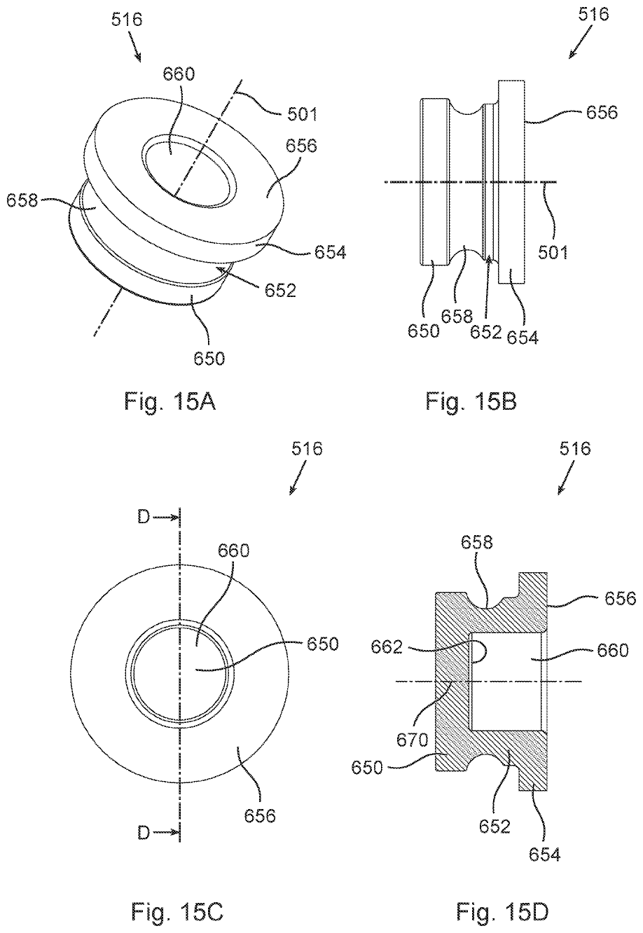

Reference is now made to FIGS. 15A-15D , which are simplified respective perspective, planar side view, planar top view and sectional illustration taken along lines D-D in FIG. 15C of the male seal element 516, forming part of the first valve assembly 502 of the breakaway intra-medical tubing connector assembly 500 of FIGS. 12A and 12B.

The male seal element 516 is arranged about longitudinal axis 501 and is preferably integrally molded, and preferably made of plastic, such as TPE, Polyethylene or any other suitable resilient plastic material.

It is seen in FIGS. 15A-15D that the male seal element 516 is generally cup-shaped and has a generally circular sealing portion 650 and a generally cylindrical portion 652 extending rearwardly therefrom, transversely with respect thereto. An annular flange 654 extends radially outwardly from the rearward end of the cylindrical portion 652 and defines a rearwardmost annular edge 656. Typically, a circular groove 658 is formed on the outer surface of the cylindrical portion 652 and serves as the fulcrum point of the male seal element 516 allowing its elastic deformation upon exertion of compressive pressure thereon.

A socket 660 is formed within the male seal element 516 and extends from the rearwardmost annular edge 656 to a rearwardly facing surface 662.

A normally closed slit 670 is formed through the circular sealing portion 650.

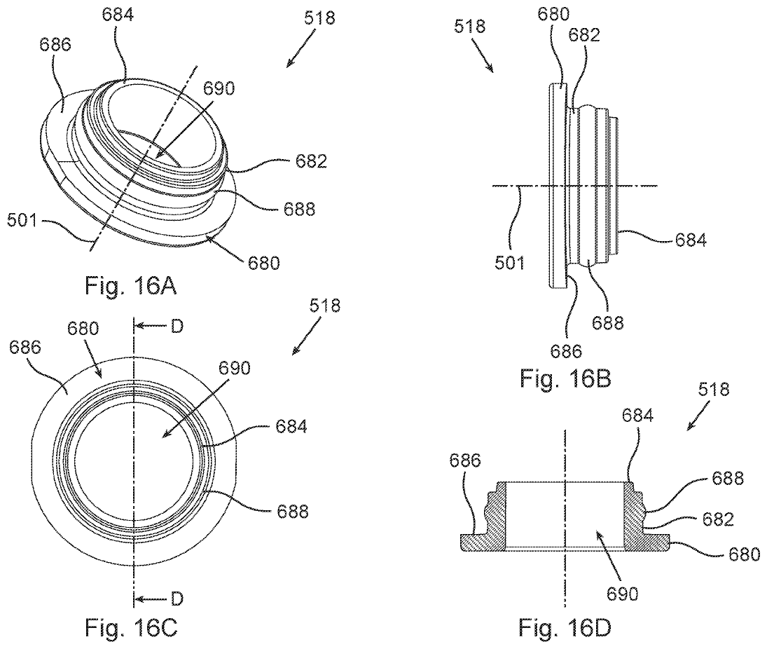

Reference is now made to FIGS. 16A-16D , which are simplified respective perspective, planar side view, planar top view and sectional illustration taken along lines D-D in FIG. 16C of the male seal lock 518, forming part of the first valve assembly 502 of the breakaway intra-medical tubing connector assembly 500 of FIGS. 12A and 12B.

The male seal lock 518 is arranged about the longitudinal axis 501 and is preferably an integrally molded element.

It is seen in FIGS. 16A-16D that the male seal element 518 has a generally annular flange portion 680 and a generally cylindrical portion 682 extending rearwardly therefrom, transversely with respect thereto and terminating at a rearwardmost annular edge 684. The annular flange portion 682 defines a rearwardly facing shoulder 686 extending radially outwardly with respect to the cylindrical portion 682.

An annular protrusion 688 is formed on the outer surface of the cylindrical portion 682 and preferably disposed at an intermediate location along the longitudinal extent thereof. A bore 690 is formed through the male seal element 518.

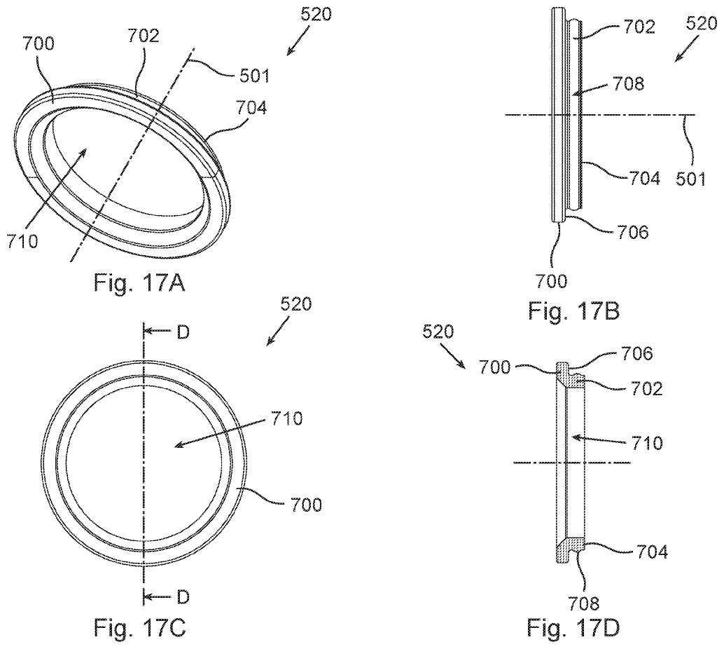

Reference is now made to FIGS. 17A-17D , which are simplified respective perspective, planar side view, planar top view and sectional illustration taken along lines D-D in FIG. 17C of the stopping ring 520, forming part of the first valve assembly 502 of the breakaway intra-medical tubing connector assembly 500 of FIGS. 12A and 12B.

The stopping ring 520 is arranged about the longitudinal axis 501 and is preferably an integrally molded element.

It is seen in FIGS. 17A-17D that the stopping ring 520 has a generally annular flange portion 700 and a generally cylindrical portion 702 extending rearwardly therefrom, transversely with respect thereto and terminating at a rearwardmost annular edge 704. The annular flange portion 700 defines a rearwardly facing shoulder 706 extending radially outwardly with respect to the cylindrical portion 702.

An annular protrusion 708 is formed on the outer surface of the cylindrical portion 702 and preferably disposed at an intermediate location along the longitudinal extent thereof. A bore 710 is formed through the stopping ring 520.

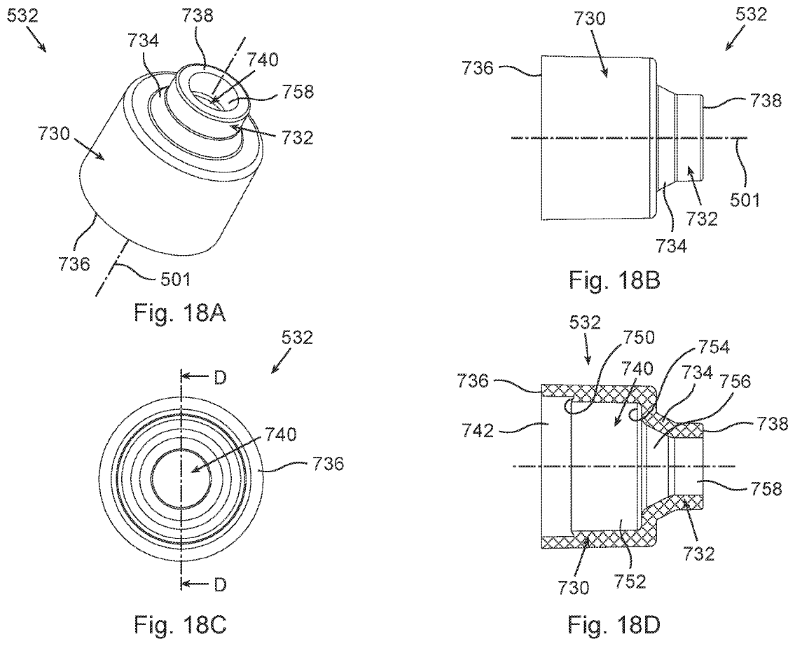

Reference is now made to FIGS. 18A-18D , which are simplified respective perspective, planar side view, planar top view and sectional illustration taken along lines D-D in FIG. 18C of the female seal cover 532, forming part of the second valve assembly 504 of the breakaway intra-medical tubing connector assembly 500 of FIGS. 12A and 12B.

The female seal cover 532 is arranged along the longitudinal axis 501 and is preferably an integrally molded element. The female seal element 532 preferably includes a forward cylindrical portion 730 and a rearward neck portion 732 disposed rearwardly therefrom and having a generally smaller outer diameter with respect to the forward cylindrical portion 730. A rearwardly tapered portion 734 joins the forward cylindrical portion 730 with the rearward neck portion 732. The forward cylindrical portion 730 defines a forwardmost circumferential edge 736 and the rearward neck portion 732 defines a forwardmost circumferential edge 738.

A through bore 740 is formed through the female seal element 532 and typically comprises a forward portion 742 extending from the forwardmost circumferential edge 736 to a forwardly facing shoulder 750, an intermediate portion 752 extending from the forwardly facing shoulder 750 to a forwardly facing surface 754 disposed adjacent the rearwardly tapered portion 734. The through bore 740 further comprises a tapered rearward portion 756 and a rearward portion 758 terminating adjacent the forwardmost circumferential edge 738.

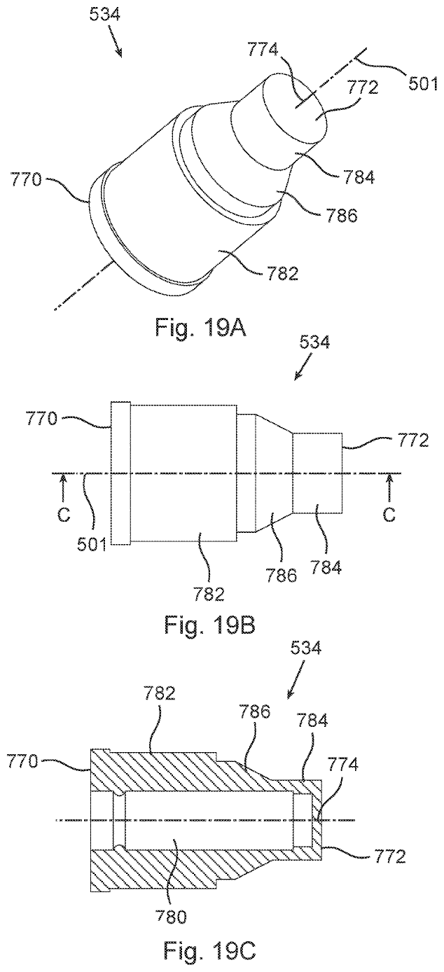

Reference is now made to FIGS. 19A-19C , which are simplified respective perspective, planar side view and sectional illustration taken along lines C-C in FIG. 19B of the female seal element 534, forming part of the second valve assembly 504 of the breakaway intra-medical tubing connector assembly 500 of FIGS. 12A and 12B.

It is appreciated that the female seal element 534 is essentially similar to various commercially available seal elements from various manufacturers, such as Haemopharm, Halkey-Roberts, NP Medical.

The female seal element 534 is preferably resilient and compressible and has a forward open end 770 and a rearward selectably openable end 772, including a selectively openable slit 774. The rearward selectably openable end 772 of the female seal element 534 is disposed in its normally closed operative orientation at rest, when the female seal element 534 is not compressed. It is noted that the female seal element 534 is biased to its normally closed operative orientation when no pressure is applied thereupon. A fluid flow passage 780 is defined by the inner volume of the female seal element 534. The rearward selectably openable end 772 provides a swabbable surface that may be cleaned by the physician in order to prevent contamination. The female seal element 534 includes a generally cylindrical portion 782 adjacent the forward open end 770 thereof, a rearward generally cylindrical portion 784 adjacent the rearward selectably openable end 772, having a diameter that is generally smaller with respect to the forward cylindrical portion 782 and a tapered portion 786 joining the two cylindrical portions 782 and 784.

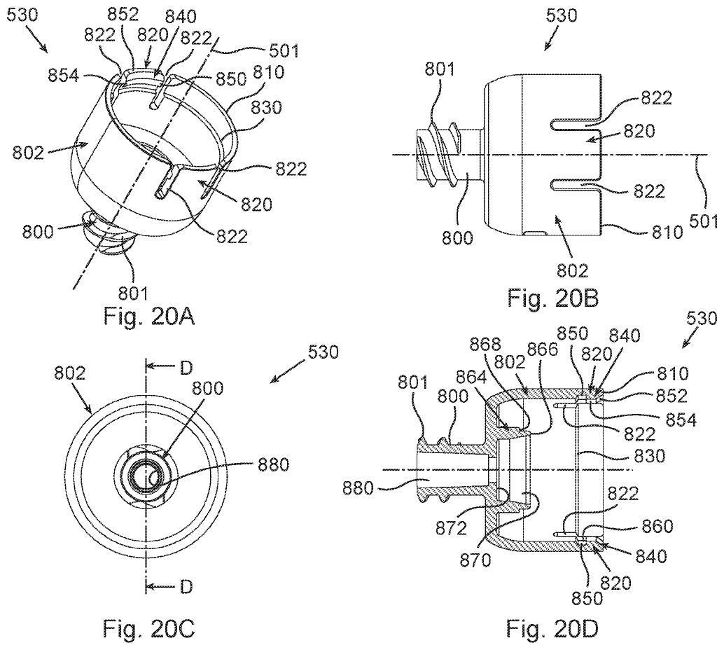

Reference is now made to FIGS. 20A-20D, which are simplified respective perspective, planar side view, planar top view and a sectional illustration taken along lines D-D in FIG. 20C of the female connector 530, forming part of the second valve assembly 504 of the breakaway intra-medical tubing connector assembly 500 of FIGS. 12A and 12B.

The female connector 530 has a female luer portion 800 with an external threading 801 and a hollow cup-shaped rearward connector portion 802 extending rearwardly therefrom to a rearwardmost end surface 810.

Typically, two diametrically opposed snap portions 820 are formed generally at the rearward end of the connector portion 802, extending rearwardly from the rearwardmost end surface 810. Each snap portion 820 is formed by two radially spaced grooves 822 extending axially forwardly from the rearwardmost end surface 810 to a generally intermediate location along the longitudinal extent of the connector portion 802. It is noted that that any other number of snap portions 820 can be formed on the connector portion 802 as part of an embodiment of the present invention.

It is seen in FIGS. 20A and 20D that a circumferential rearwardly facing shoulder 830 is formed on the inner surface of the connector portion 802 and is disposed forwardly of the reawardmost end surface 810, so that inner diameter of the connector portion 802 rearwardly of the rearwardly facing shoulder 830 is larger than the inner diameter of the connector portion 802 forwardly of the rearwardly facing shoulder 830.

It is specifically seen that radially inwardly extending protrusions 840 are formed at the rearward ends of the snap portions 820, extending radially inwardly from the rearwardmost end surface 810, thereby forming a radial groove 850 between the protrusions 840 and the rearwardly facing shoulder 830.

It is noted that alternatively, the inwardly extending protrusion 840 may extend around the entire circumference of the rearward end of the connector portion 802 to form a circumferential groove 850 along the entire circumference of the connector portion 802.

It is further noted that alternatively the radially spaced grooves 822 may be obviated and the rearwardmost end surface 810 may therefore be a circular continuous surface, from which extends the inwardly extending protrusion 840.

It is particularly seen that the radially extending protrusion 840 has a forwardly tapered surface 852 extending from the rearwardmost end surface 810, a side surface 854 extending axially forwardly therefrom and a forwardly facing shoulder 860 extending generally transversely with respect to the longitudinal axis 501.

A cup-shaped protrusion 864 is arranged co-axially with the connector portion 802 and extends rearwardly from the forward end of the connector portion 802 to a circumferential rearward end surface 866. A rearwardly facing shoulder 868 is formed on the outer surface of the protrusion 864 and is slightly forwardly spaced from the rearward end surface 866. The cup-shaped protrusion 864 has a socket 870 defining a rearwardly facing surface 872 and fluidly communicating with a bore 880 formed through the female luer portion 800.

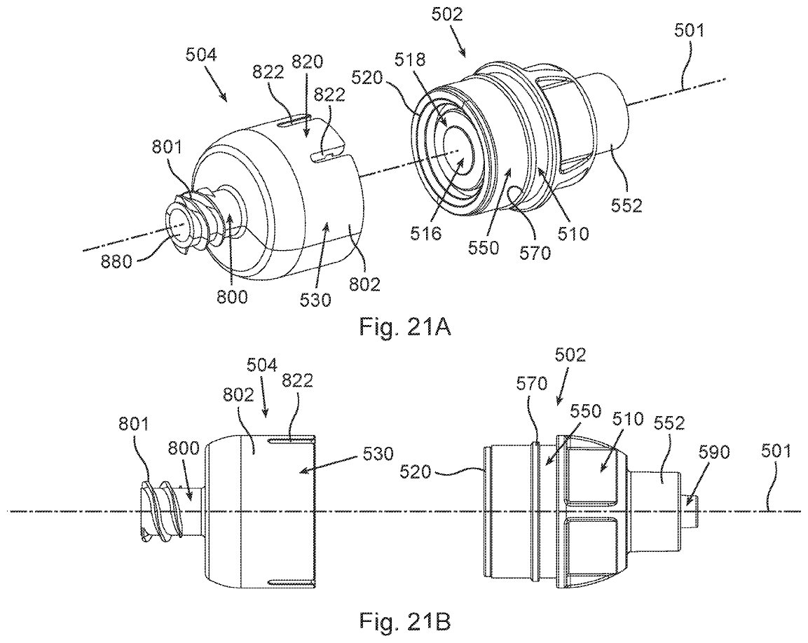

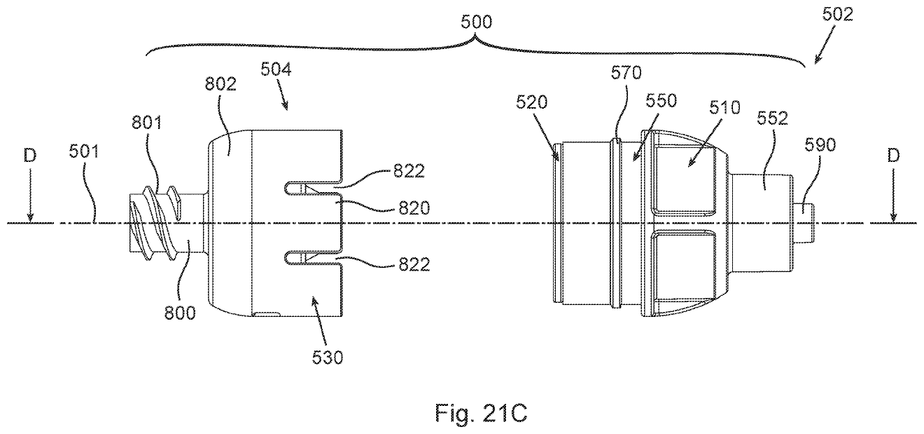

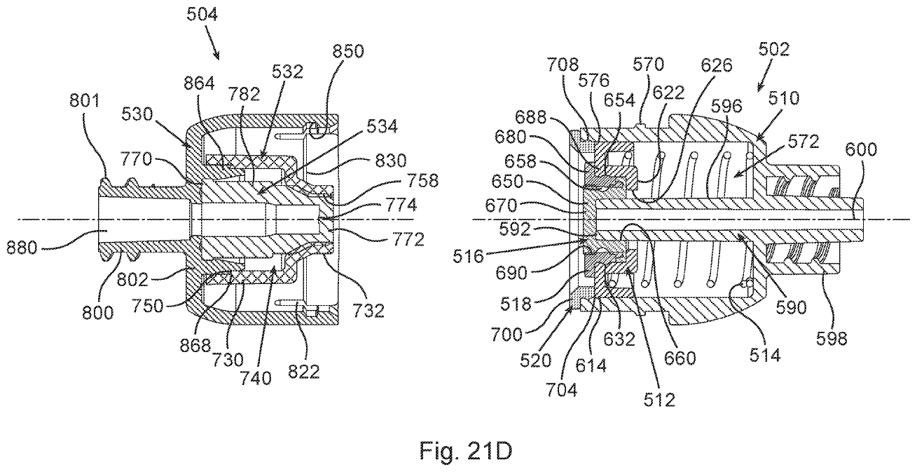

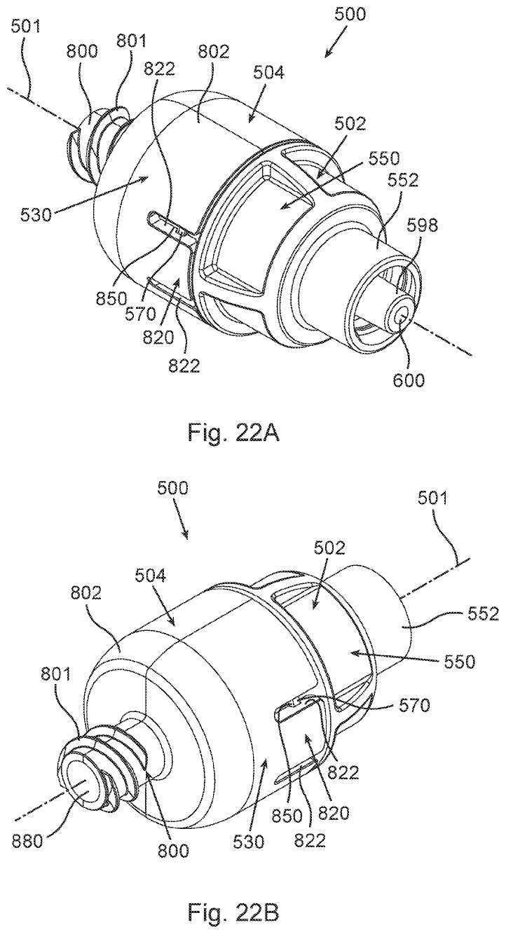

Reference is now made to FIGS. 21A-21D, which are simplified respective perspective, two different planar side views and a sectional illustration taken along lines D-D in FIG. 21C of the breakaway intra-medical tubing connector assembly 500 of FIGS. 12A and 12B, shown in a disconnected operative orientation, when the first valve assembly 502 and the second valve assembly 504 are disconnected.

It is seen in FIGS. 21A-21D that the first valve assembly 502 and the second valve assembly 504 are disconnected from each other and their respective seal elements, namely the male seal element 516 and the female seal element 534 are sealingly disposed in their normally closed operative orientation.

It is specifically seen in FIG. 21D that the male seal element 516 is fixedly retained between the shuttle 512 and the male seal lock 518. It is further seen that a spring 514 is supported between the male connector 510 and the shuttle 512 and thereby biases the shuttle 512 along with the male seal element 516 and the male seal lock 518 forwardly up to engagement of the shuttle 512 with the stopping ring 520, which is fixedly attached to the forward end of the male connector 510.

It is noted that the shuttle 512 with the male seal element 516 and the male seal lock 518 biased by the spring 514 are all disposed within the socket 572 of the male connector 510 and retained therewithin by the stopping ring 520.

It is a particular feature of an embodiment of the present invention that the male seal element 516 engages the forward edge 592 of the forward cannula portion 596 of the male connector 510 in this disconnected operative orientation, such that the sealing portion 650 of the male seal element 516 seats across the fluid flow path 600 of the cannula 590 of the male connector 510 with the slit 670 closed and thus seals the fluid flow passage therethrough.

The sealing portion 650 of the male seal element 516 provides a swabbable surface that may be cleaned by the physician in order to prevent contamination.

It is noted that the male seal element 516 is normally closed at rest, unless penetrated by the cannula 590 of the male connector 510.

It is a particular feature of an embodiment of the present invention that the shuttle 512 along with the male seal element 516 is axially moveable relative to the male connector 510 and thus relative to the cannula 590 of the male connector 510 and is biased forwardly under the urge of the spring 514. In this disconnected operative orientation, the shuttle 512 is disposed in its forwardmost position and its further forward axial displacement is prevented by engagement with the stopping ring 520. Specifically, the circular flange 614 of the shuttle 512 engages the annular edge 704 of the stopping ring 520 in this forward position of the shuttle 512. The stopping ring 520 is fixedly attached to the male connector 510 by means of engagement between the circumferential groove 576 of the male connector 510 and the annular protrusion 708 of the stopping ring 520.

It is particularly seen in FIG. 21D that the spring 514 is supported between the circular flange 554 of the male connector 510 and the circular flange 614 of the shuttle 512.

It is further seen in FIG. 21D that the male seal element 516 is seated between the shuttle 512 and the male seal element 518 such that the annular flange 654 of the male seal element is supported between the forwardly facing shoulder 624 of the shuttle 512 and the rearwardmost annular edge 684 of the male seal lock 518. The male seal lock 518 is fixedly attached to the shuttle 512 by means of engagement of the annular protrusion 688 of the male seal lock 518 with the circumferential groove 632 of the shuttle 512. Thus, the shuttle 512, the male seal element 516 and the male seal lock 518 are axially displaceable together as one unit.

It is further seen in FIG. 21D that the female seal element 534 is fixedly retained between the female connector 530 and the female seal cover 532, whereas the female seal cover 532 is fixedly attached to the female connector 530 by means of engagement between the forwardly facing shoulder 750 of the female seal cover 532 and the rearwardly facing shoulder 868 of the female connector 530.

The female seal element 534 is seated partially within the socket 870 of the cup-shaped protrusion 864 of the female connector 530 and partially within the trough bore 740 of the female seal cover 532. It is specifically seen that the cylindrical portion 784 of the female seal element 534 is seated within the rearward portion 758 of the trough bore 740 of the female seal cover 532 and is disposed in its normally closed operative orientation such that slit 774 is sealed and fluid flow passage into bore 880 is prevented.

The selectably openable end 772 of the female seal element 534 provides a swabbable surface that may be cleaned by the physician in order to prevent contamination.

It is noted that the female seal element 534 is biased to its normally closed position once a compression force exerted thereon is removed.

The female seal element 534 is supported against the female connector 530 by engagement of the forward open end 770 of the female seal element 534 with the forward end of the connector portion 802 of the female connector 530.

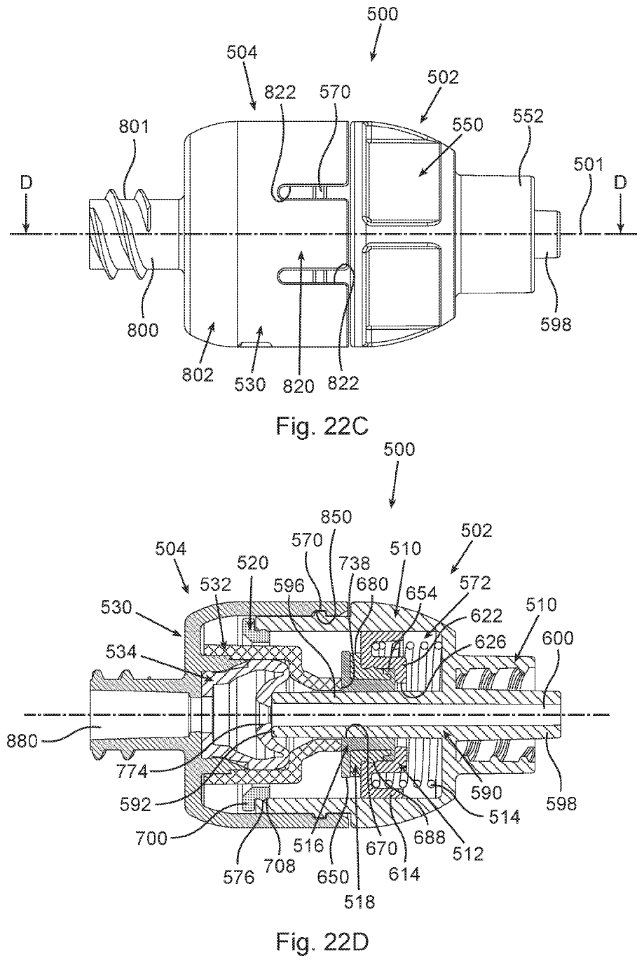

Reference is now made to FIGS. 22A-22D, which are simplified respective two perspective views taken from opposite directions, a planar side view and a sectional illustration taken along lines D-D in FIG. 22C of the breakaway intra-medical tubing connector assembly 500 of FIGS. 12A and 12B, shown in a connected operative orientation, when the first valve assembly 502 and the second valve assembly 504 are connected.

It is seen in FIGS. 22A-22D that the first valve assembly 502 and the second valve assembly 504 are connected to each other and their respective seal elements, namely the male seal element 516 and the female seal element 534 are now open and allow fluid flow passage through the cannula 590 of the male connector 510 of the first valve assembly 502, via the opened slit 774 and the fluid flow passage 780 of the female seal element 534 and into bore 880 of the female connector 530 of the second valve assembly 504.

It is appreciated that all spatial relationships between the various components of the breakaway intra-medical tubing connector assembly 500 remain the same as described hereinabove with respect to the disconnected operative orientation illustrated in FIGS. 22A-22D, besides the following spatial relationships:

It is a particular feature of an embodiment of the present invention, as particularly seen in FIGS. 22A-22D, that upon connection of the first valve assembly 502 and the second valve assembly 504, the male connector 510 and the female connector 530 are selectably attached to each other in a tensile force responsive disconnectable snap-fit manner. Specifically, during axial displacement of the first valve assembly 502 and the second valve assembly 504 in opposite axial directions, the coupling snap 570 of the male connector 510 engages the radial groove 850 of protrusions 840 of the female connector 530 and causes momentary radial outward deflection of the snap portions 820 of the female connector 530, thus the coupling snap 570 of the male connector 510 skips over the protrusions 840 and is seated within the radial groove 850 of the female connector 530. The remaining circumferential surface of the coupling snap 570 of the male connector 510 rearwardly abuts the rearwardly facing shoulder 830 of the female connector 530.

It is noted that the tensile force that is required in order to disconnect the first valve assembly 502 and the second valve assembly 504 is typically in the range of 0.5-3 kgf.

It is a further particular feature of an embodiment of the present invention that in this connected operative orientation of the breakaway intra-medical tubing connector assembly 500, when the first valve assembly 502 and the second valve assembly 504 are attached to each other in a tensile force responsive disconnectable snap-fit manner, the cannula 590 of the male connector 510 causes opening of both the male seal element 516 and the female seal element 534 and thereby allows fluid flow passage through the first valve assembly 502 and the second valve assembly 504, specifically through the cannula 590 of the male connector 510 of the first valve assembly 502, via the opened slit 774 and the fluid flow passage 780 of the female seal element 534 and into bore 880 of the female connector 530 of the second valve assembly 504.

It is a still further particular feature of an embodiment of the present invention that upon connection of the first valve assembly 502 and the second valve assembly 504, a portion of the second valve assembly 504 urges rearward axial displacement of the shuttle 512 relative to the cannula 590 of the male connector 510 against the urge of the spring 514 and thereby causing opening of the male seal element 516 and the female seal element 534 by the cannula 590, permitting fluid flow passage from the first valve assembly 502 to the second valve assembly 504 or vice-versa.

As specifically seen in FIG. 22D, upon connection of the first valve assembly 502 and the second valve assembly 504 the forwardmost circumferential edge 738 of the female seal cover 532 engages the sealing portion 650 of the male seal element 516 and pushes the shuttle 512, along with the male seal element 516 and the male seal lock 518 axially rearwardly against the urge of the spring 514. It is seen in FIG. 22D that the spring 514 is now compressed and the forward end 596 of the cannula 590 of the male connector 510 penetrates the sealing portion 650 of the male seal element 516 through slit 670 and enters the rearward portion 758 of the through bore 740 of the female seal cover 532. Upon insertion of the forward end 596 of the cannula 590 of the male connector 510 into the through bore 740 of the female seal cover 532, the female seal element 534 is axially compressed and thus buckles and provides for opening of the slit 774. Once the cannula 590 of the male connector 510 penetrates the slit 670 of the male seal element 516 and causes opening of the slit 774 of the female seal element 534, fluid flow passage through the two valve assemblies 502 and 504 is established.

It is noted that the female luer portion 800 of the female connector 530 may be connected to a first medical tube and the rearward end 598 of the cannula 590 of the male connector 510 may be connected to a second medical tube, which may be adapted to be positioned in a certain treatment area within the patient's body, thus dis-location of one of the medical tubes from its desired position may require replacement of the entire medical set in absence of a connector such as the breakaway intra-medical tubing connector assembly 500 constructed and operative in accordance with an embodiment of the present invention.

It is noted that the fluid flow direction can be established in an opposite direction.

It is noted that compression forces exerted on both the male seal element 516 and the female seal element 534 by the cannula 590 are enabled by the fact that the first valve assembly 502 and the second valve assembly 504 are held in place relative to each other due to snap-fit connection between the male connector 510 and the female connector 530.

It is a particular feature of an embodiment of the present invention that upon application of tensile force in a direction of disconnection of the first valve assembly 502 and the second valve assembly 504, the coupling snap 570 of the male connector 510 disengages the radial groove 850 of the female connector 530 and thus the cannula 590 of the male connector 510 is removed from the female seal cover 532, thereby causing sealing of the female seal element 534, due to the fact that it is biased to its normally closed position. Further, upon disengagement between the female seal cover 532 and the shuttle 512, through the male seal element 516, the spring 514 biases the shuttle 512, the male seal element 516 and the male seal lock 518 to move axially forwardly up to sealing of the slit 670 of the male seal element 516.

It is noted that the shuttle 512 is biased forwardly up to engagement thereof with the stopping ring 520 and upon disconnection of the first valve assembly 502 and the second valve assembly 504, the intra-medical tubing connector assembly 500 is positioned in its disconnected operative orientation again, as illustrated in FIGS. 21A-21D, where no fluid flow passage is allowed through the valve assemblies 502 and 504.

It is a particular feature of an embodiment of the present invention that upon disconnection of the first and second valve assemblies 502 and 504, both of the respective seal elements 516 and 534 are automatically bidirectionally closed, thus preventing fluid flow passage from the two medical tubes.

It is noted that tensile force exerted on a medical set such as IV line may be unintentional, but due to the presence of the breakaway intra-medical tubing connector assembly 500, the catheter or other medical tube associated with at least one of the luer-actuated valve assemblies 502 and 504 remains in its position within the treatment site and the luer-actuated valve assemblies 502 and 504 are safely sealed once disconnected from each other. Therefore, no fluid can unintentionally flow out of the IV line and risk of contamination of the treatment site is prevented due to sealing of the luer-actuated valve assemblies 502 and 504 and the ability to clean the exterior surface of the sealing elements 516 and 534 thereof.

It will be appreciated by persons skilled in the art that the present invention is not limited by what has been particularly shown and described hereinabove. Rather the scope of the present invention includes both combinations and subcombinations of various features described hereinabove as well as variations and modifications thereof which are not in the prior art.

1. An automatically bidirectionally-sealable safe disconnect intra-medical tubing connector assembly comprising:
first and second luer-actuated valve assemblies configured for connection to opposite intermediate ends of a medical tube and configured to be connected to each other in a snap-fit manner and arranged for tensile force responsive disconnection;
said first luer-actuated valve assembly comprises a cannula defining a fluid flow path and a first seal element retained within a shuttle element, wherein said shuttle element is configured to be axially displaceable relative to said cannula and further configured to be biased forwardly by a biasing element;
said second luer-actuated valve assembly comprises a second seal element.

2. The automatically bidirectionally-sealable safe disconnect intra-medical tubing connector assembly of claim 1 and wherein connection of said first luer-actuated valve assembly and said second luer-actuated valve assembly provides for opening of both said first and second seal elements and establishing of fluid flow through said fluid flow path and wherein disconnection of said first luer-actuated valve assembly and said second luer-actuated valve assembly provides for automatic closing of both said first and second sealing elements.

3. The automatically bidirectionally-sealable safe disconnect intra-medical tubing connector assembly of claim 1 and wherein when said first and second luer-actuated valve assemblies are disconnected, said shuttle element is disposed in a forward position, such that the first sealing element is closed and wherein when said first and second luer-actuated valve assemblies are connected, the second luer-actuated valve assembly urges displacement of said shuttle element relative to said cannula against the urge of said biasing element, thereby opening both the first and the second seal elements.

4. The automatically bidirectionally-sealable safe disconnect intra-medical tubing connector assembly of claim 1 and wherein said connector assembly is configured to assume a first and a second operative orientation:
said first operative orientation in which said first and second luer-actuated valve assemblies are connected in a snap-fit manner and said cannula is configured to open both the first seal element and the second seal element and thereby establish fluid communication between the first and second luer actuated valve assemblies; and
said second operative orientation in which said first and second luer-actuated valve assemblies are disconnected following exertion of axial tensile force on the first and second luer-actuated valve assemblies in two different axial directions, and said first seal element and the second seal element are biased to their closed position, thereby preventing fluid communication between the first and second luer actuated valve assemblies.

5.-7. (canceled)

8. The automatically bidirectionally-sealable safe disconnect intra-medical tubing connector assembly of claim 1 and wherein each of said first seal element and said second seal element has a first open end and a second selectably openable end, including a selectively openable slit and wherein said first seal element and said second seal element is biased to its normally closed position when no compression force is exerted thereon.

9. The automatically bidirectionally-sealable safe disconnect intra-medical tubing connector assembly of claim 1 and wherein tensile force in the range of 0.5 kgf-3 kgf is required in order to disconnect said first luer-actuated valve assembly from said second luer-actuated valve assembly.

10. (canceled)

11. The automatically bidirectionally-sealable safe disconnect intra-medical tubing connector assembly of claim 1 and wherein the first luer-actuated valve assembly comprises a first housing which is integrally made or fixedly connected to said cannula, said shuttle being slidably seated within said first housing and said first seal element is retained within said shuttle by a male seal lock, which is fixedly coupled with said shuttle, such that in a closed position of the first seal element, said first seal element provides for a fluid tight sealing relative to the first housing.

12. The automatically bidirectionally-sealable safe disconnect intra-medical tubing connector assembly of claim 1 and wherein engagement of said shuttle with said first housing, limits forward axial displacement of the shuttle relative to the first housing.

13. The automatically bidirectionally-sealable safe disconnect intra-medical tubing connector assembly of claim 1 and wherein the second luer-actuated valve assembly comprises a second housing.

14.-19. (canceled)

20. An automatically bidirectionally-sealable safe disconnect intra-medical tubing connector assembly comprising:
first and second luer-actuated valve assemblies configured for connection to opposite intermediate ends of a medical tube and configured to be connected to each other in a snap-fit manner and arranged for tensile force responsive disconnection;
said first luer-actuated valve assembly comprises a first housing and a cannula defining a fluid flow path and a first seal element retained within a shuttle element, wherein said shuttle element is configured to be axially displaceable relative to said cannula;
said second luer-actuated valve assembly comprises a second housing and a second seal element, and wherein said first housing and said second housing are configured to be connected to each other in a snap-fit manner.

21. The automatically bidirectionally-sealable safe disconnect intra-medical tubing connector assembly of claim 20 and wherein said shuttle element is configured to be axially displaceable relative to said cannula and further configured to be biased forwardly by a biasing element.

22. The automatically bidirectionally-sealable safe disconnect intra-medical tubing connector assembly of claim 20 and wherein connection of said first luer-actuated valve assembly and said second luer-actuated valve assembly provides for opening of both said first and second seal elements and establishing of fluid flow through said fluid flow path and wherein disconnection of said first luer-actuated valve assembly and said second luer-actuated valve assembly provides for automatic closing of both said first and second sealing elements.

23. The automatically bidirectionally-sealable safe disconnect intra-medical tubing connector assembly of claim 21 and wherein when said first and second luer-actuated valve assemblies are disconnected, said shuttle element is disposed in a forward position, such that the first sealing element is closed and wherein when said first and second luer-actuated valve assemblies are connected, the second luer-actuated valve assembly urges displacement of said shuttle element relative to said cannula against the urge of said biasing element, thereby opening both the first and the second seal elements.

24. The automatically bidirectionally-sealable safe disconnect intra-medical tubing connector assembly of claim 20 and wherein said connector assembly is configured to assume a first and a second operative orientation:
said first operative orientation in which said first and second luer-actuated valve assemblies are connected in a snap-fit manner and said cannula is configured to open both the first seal element and the second seal element and thereby establish fluid communication between the first and second luer actuated valve assemblies; and
said second operative orientation in which said first and second luer-actuated valve assemblies are disconnected following exertion of axial tensile force on the first and second luer-actuated valve assemblies in two different axial directions, and said first seal element and the second seal element are biased to their closed position, thereby preventing fluid communication between the first and second luer actuated valve assemblies.

25.-27. (canceled)

28. The automatically bidirectionally-sealable safe disconnect intra-medical tubing connector assembly of claim 20 and wherein each of said first seal element and said second seal element has a first open end and a second selectably openable end, including a selectively openable slit and wherein said first seal element and said second seal element is biased to its normally closed position when no compression force is exerted thereon.

29.-31. (canceled)

32. The automatically bidirectionally-sealable safe disconnect intra-medical tubing connector assembly of claim 20 and wherein engagement of said shuttle with said first housing, limits forward axial displacement of the shuttle relative to the first housing.

33. The automatically bidirectionally-sealable safe disconnect intra-medical tubing connector assembly of claim 20 and wherein said second housing is integrally made or fixedly connected to a female seal cover and forming an interior volume therebetween, and wherein said second seal element is seated within the interior volume in a fluid tight sealing manner relative to the second housing and to the female seal cover.

34. (canceled)

35. The automatically bidirectionally-sealable safe disconnect intra-medical tubing connector assembly of claim 20 and wherein first coupling snaps are arranged on said first housing and second coupling snaps are arranged on said female seal cover, and wherein said first luer-actuated valve assembly and said second luer-actuated valve assembly are connected due to engagement of said first coupling snaps and said second coupling snaps.

36. The automatically bidirectionally-sealable safe disconnect intra-medical tubing connector assembly of claim 20 and wherein during axial displacement of the first luer-actuated valve assembly and the second luer-actuated valve assembly in opposite axial directions, the first coupling snaps disengage the second coupling snaps, causing disconnection of the first luer-actuated valve assembly from the second luer-actuated valve assembly.

37. The automatically bidirectionally-sealable safe disconnect intra-medical tubing connector assembly of claim 20 and wherein first coupling snaps are arranged on said first housing and second coupling snaps are arranged on said second housing, and wherein said first luer-actuated valve assembly and said second luer-actuated valve assembly are connected due to engagement of said first coupling snaps and said second coupling snaps.

38. (canceled)
